(19) United States
(12) Patent Application Publication
(10) Pub. No.: US 2021/0147906 A1
(43) Pub. Date: May 20, 2021

(54) DETECTION DETERMINING METHOD, DETECTION DETERMINING DEVICE, NON-TRANSITORY RECORDING MEDIUM STORING DETECTION DETERMINING PROGRAM, AND DEVICE

(71) Applicants: Yusuke OSAKI, Tokyo (JP); Satoshi IZUMI, Tokyo (JP); Yudai KAWASHIMA, Kanagawa (JP); Michie HASHIMOTO, Tokyo (JP); Manabu SEO, Kanagawa (JP); Koei SUZUKI, Kanagawa (JP); Hirotaka UNNO, Tokyo (JP)

(72) Inventors: Yusuke OSAKI, Tokyo (JP); Satoshi IZUMI, Tokyo (JP); Yudai KAWASHIMA, Kanagawa (JP); Michie HASHIMOTO, Tokyo (JP); Manabu SEO, Kanagawa (JP); Koei SUZUKI, Kanagawa (JP); Hirotaka UNNO, Tokyo (JP)

(21) Appl. No.: 16/766,117

(22) PCT Filed: Nov. 22, 2018
(86) PCT No.: PCT/JP2018/043275

§ 371 Date: May 21, 2020

(30) Foreign Application Priority Data
Nov. 24, 2017 (JP) 2017-226084
Jun. 14, 2018 (JP) 2018-113937
Nov. 21, 2018 (JP) 2018-218544

(51) Int. Cl. C12Q 1/6806; B01L 3/02; C12Q 1/689; C12Q 1/70

(52) U.S. Cl. CPC C12Q 1/6806; B01L 3/0268; C12Q 1/689; C12Q 1/70; B01L 2200/061; C12Q 2565/125; B01L 2200/143; B01L 2400/0439; C12Q 2531/113; C12Q 2537/16; B01L 2200/0647

(57) ABSTRACT

Provided is a detection determining method used in detection of a testing target in a sample by amplification of the testing target and an amplifiable reagent, wherein the amplifiable reagent is provided in a number of 200 or less, the detection determining method including a determining step of determining that the testing target is present and a detection result is positive when the amplifiable reagent is amplified and the testing target is amplified, and determining that the testing target is absent or at least less than a specific copy number of the amplifiable reagent and a detection result is negative when the amplifiable reagent is amplified and the testing target is not amplified. Also provided are a detection determining device, a detection determining program, and a device used for the detection determining method.

### TECHNICAL FIELD

The present disclosure relates to a detection determining method, a detection determining device, a detection determining program, and a device.

### BACKGROUND ART

In recent years, increased sensitivity of analytical techniques has enabled measurement of measurement targets in unit of the number of molecules, and industrial application of gene detection techniques for detecting trace nucleic acids to foods, environmental audits, and medical care has been demanded. Particularly, detection of pathogens, viruses, or unapproved genetically modified foods is often intended for confirming absence in analyte samples, and high-level detection and determination of the detection result are demanded.

Polymerase chain reaction (PCR) methods are used in detection of pathogens and diagnoses of pathological conditions for infectious diseases, contamination tests for genetically modified crops, and genetic diagnoses in negative tests for viruses. The PCR methods are techniques for amplifying DNA stepwise, and can specifically amplify an arbitrary partial base sequence from an analyte sample. Therefore, the PCR methods are widely used in, for example, genetic testing.

In testing of an analyte sample, when a target nucleic acid is not detected from the sample, the detection result is determined as negative. However, in the case of the negative determination, problematically, it has been impossible to determine definitely whether the testing target nucleic acid is actually absent in the analyte sample, i.e., whether the negative determination is correct, or whether the nucleic acid is actually present but erroneously determined as absent (negative) due to failure of identifying, i.e., whether the detection result is false-negative.

Hence, there have been proposed various PCR testing methods for avoiding false-negative determinations.

There has been disclosed a microbial detection method of performing a PCR reaction by putting two pairs of different primer sets, namely a first pair of primers and a second pair of primers in one reaction system (for example, see PTL 1).

There has also been disclosed a DNA detection method of synthesizing DNA that is amplified by the same primer as the primer for amplifying a certain portion of target DNA and can be distinguished from DNA of the target portion by, for example, the base length, and overcoming, for example, the false-negative problem based on PCR performed by adding the synthesized DNA and PCR performed without adding the synthesized DNA (for example, see PTL 2).

### CITATION LIST

### Patent Literature

### PTL 1: Japanese Unexamined Patent Application Publication No. 2011-223940

### PTL 2: Japanese Unexamined Patent Application Publication No. 09-224699

### SUMMARY OF INVENTION

### Technical Problem

The present disclosure has an object to provide a detection determining method that, in detection of a testing target contained in a sample, provides an improved accuracy for negative determination with a capability of more securely avoiding false-negative causing situations particularly when the copy number of the testing target is low.

### Solution to Problem

According to one aspect of the present disclosure, a detection determining method is a detection determining method used in detection of a testing target in a sample by amplification of the testing target and an amplifiable reagent, wherein the amplifiable reagent is provided in a specific copy number of 200 or less. The detection determining method includes a determining step of determining that the testing target is present and a detection result is positive when the amplifiable reagent is amplified and the testing target is amplified, and determining that the testing target is absent or at least less than the specific copy number of the amplifiable reagent and a detection result is negative when the amplifiable reagent is amplified and the testing target is not amplified.

### Advantageous Effects of Invention

The present disclosure can provide a detection determining method that, in detection of a testing target contained in a sample, provides an improved accuracy for negative determination with a capability of more securely avoiding false-negative causing situations particularly when the copy number of the testing target is low.

### BRIEF DESCRIPTION OF DRAWINGS

FIG. 1 is a block diagram illustrating an example of a hardware configuration of a detection determining device.

FIG. 2 is a diagram illustrating an example of a functional configuration of a detection determining device.

FIG. 3 is a flowchart illustrating an example of a procedure of a detection determining program.

FIG. 4 is a graph plotting a relationship between a copy number having variation according to a Poisson distribution and a coefficient of variation CV.

FIG. 5 is a perspective view illustrating an example of a device of the present disclosure.

FIG. 6 is a perspective view illustrating another example of a device of the present disclosure.

FIG. 7 is a side view of FIG. 6.

FIG. 8 is a perspective view illustrating another example of a device of the present disclosure.

FIG. 9 is a diagram illustrating an example of positions of wells to be filled with an amplifiable reagent in a device of the present disclosure.

FIG. 10 is a diagram illustrating another example of positions of wells to be filled with an amplifiable reagent in a device of the present disclosure.

FIG. 11 is a graph plotting an example of a relationship between the frequency and the fluorescence intensity of cells in which DNA replication has occurred.

FIG. 12A is an exemplary diagram illustrating an example of an electromagnetic valve-type discharging head.

FIG. 12B is an exemplary diagram illustrating an example of a piezo-type discharging head.

FIG. 12C is an exemplary diagram illustrating a modified example of the piezo-type discharging head illustrated in FIG. 12B.

FIG. 13A is an exemplary graph plotting an example of a voltage applied to a piezoelectric element.

FIG. 13B is an exemplary graph plotting another example of a voltage applied to a piezoelectric element.

FIG. 14A is an exemplary diagram illustrating an example of a liquid droplet state.

FIG. 14B is an exemplary diagram illustrating an example of a liquid droplet state.

FIG. 14C is an exemplary diagram illustrating an example of a liquid droplet state.

FIG. 15 is a schematic diagram illustrating an example of a dispensing device configured to land liquid droplets sequentially into wells.

FIG. 16 is an exemplary diagram illustrating an example of a liquid droplet forming device.

FIG. 17 is a diagram illustrating hardware blocks of a control unit of the liquid droplet forming device of FIG. 16.

FIG. 18 is a diagram illustrating functional blocks of a control unit of the liquid droplet forming device of FIG. 17.

FIG. 19 is a flowchart illustrating an example of an operation of a liquid droplet forming device.

FIG. 20 is an exemplary diagram illustrating a modified example of a liquid droplet forming device.

FIG. 21 is an exemplary diagram illustrating another modified example of a liquid droplet forming device.

FIG. 22A is a diagram illustrating a case where two fluorescent particles are contained in a flying liquid droplet.

FIG. 22B is a diagram illustrating a case where two fluorescent particles are contained in a flying liquid droplet.

FIG. 23 is a graph plotting an example of a relationship between a luminance Li when particles do not overlap each other and a luminance Le actually measured.

FIG. 24 is an exemplary diagram illustrating another modified example of a liquid droplet forming device.

FIG. 25 is an exemplary diagram illustrating another example of a liquid droplet forming device.

FIG. 26 is an exemplary diagram illustrating an example of a method for counting cells that have passed through a micro-flow path.

FIG. 27 is an exemplary diagram illustrating an example of a method for capturing an image of a portion near a nozzle portion of a discharging head.

FIG. 28 is a graph plotting a relationship between a probability P (>2) and an average cell number.

FIG. 29A is a diagram illustrating a result of agarose electrophoresis of a sample (1) performed after PCR amplification of the sample in a negative test for norovirus in a shellfish in Example 2, where the sample (1) is prepared on a 96-well plate by discharging 10 cells (copies) of 600G yeast by IJ and adding a norovirus-containing sample to the resultant (Example of the present disclosure).

FIG. 29B is a diagram illustrating a result of agarose electrophoresis of a sample (2) performed after PCR amplification of the sample, where the sample (2) is prepared to contain norovirus only.

FIG. 29C is a diagram illustrating a result of agarose electrophoresis of a sample (3) performed after PCR amplification of the sample in a negative test for norovirus in a shellfish in Example 2, where the sample (3) is prepared on a 96-well plate by diluting 600G plasmid, dispensing the resultant by an amount corresponding to 10 copies per well by a manual operation, and adding a norovirus-containing sample to the resultant (Comparative Example to IJ).

### DESCRIPTION OF EMBODIMENTS

PTL 1 mentioned above performs detection of a target gene by putting two pairs of different primer sets in one reaction system, in order to ensure success or failure of the experiment process and avoid a false-negative determination due to failure of the experiment. However, PTL 1 does not prescribe the copy number of reference DNA used as control. Hence, what is ensured is only success or failure of the experiment process, and false-negative due to any other cause, for example, due to a case where the target gene is lower than or equal to the limit of detection of the experiment process (to be described in detail below), cannot be avoided. That is, PTL 1 is not sufficient as a testing method that can more securely avoid false-negative causing situations.

For example, when an analyte sample of a very small amount is used (i.e., when the copy number of the testing target contained in the sample is low), it is impossible to determine definitely which of the cases described below is pertinent to a result of non-detection of the testing target and a determination that “the testing target is absent” (negative). That is, with the method of PTL 1, it is impossible to determine definitely whether the testing target is absent in the analyte sample (negative) or whether the testing target is present but erroneously determined as negative due to failure of identifying (false-negative).

PTL 2 mentioned above synthesizes DNA that is amplified by the same pair of primers as used for target DNA but can be distinguished from the target DNA by, for example, the base length or base sequence. PTL 2 attempts to avoid a false-negative determination based on PCR performed by adding the synthesized DNA and PCR performed without adding the synthesized DNA. However, PTL 2 does not prescribe the copy number of the synthesized DNA used for reference. Hence, PTL 2 is not sufficient as a testing method that can more securely avoid false-negative. For example, when an analyte sample of a very small amount is used (i.e., when the copy number of the testing target contained in the sample is low), it is impossible to determine definitely which of the cases described below is pertinent to a result of non-detection of the testing target and a determination that “the testing target is absent” (negative). That is, with the method of PTL 2, it is impossible to determine definitely whether the testing target is absent in the analyte sample (negative) or whether the testing target is present but erroneously determined as negative due to failure of identifying (false-negative).

Hence, the present disclosure provides a testing method that can provide an improved accuracy for negative determination by more securely avoiding false-negative causing situations even when an analyte sample of a very small amount is used (i.e., when the copy number of the testing target is low).

The present disclosure uses a device including wells into each of which an amplifiable reagent in a specific copy number is dispensed at a certain accuracy and with a coefficient of variation of higher than or equal to a certain level.

The present disclosure provides a detection determining method that is used in detection of a testing target in a sample by amplification of the testing target and an amplifiable reagent. The amplifiable reagent is provided in a specific copy number of 200 or less. The detection determining method includes a determining step of determining that the testing target is present and a detection result is positive when the amplifiable reagent is amplified and the testing target is amplified, and determining that the testing target is absent or at least less than the specific copy number of the amplifiable reagent and a detection result is negative when the amplifiable reagent is amplified and the testing target is not amplified.

The present disclosure can provide a detection determining method that, in detection of a testing target contained in a sample, can provide an improved accuracy for negative determination with a capability of more securely avoiding false-negative causing situations particularly when the copy number of the testing target is low.

(Detection Determining Method, Detection Determining Device, and Detection Determining Program)

A detection determining method of the present disclosure is a detection determining method used in detection of a testing target in a sample by amplification of the testing target and an amplifiable reagent. The amplifiable reagent is provided in a specific copy number of 200 or less. The detection determining method includes a determining step of determining that the testing target is present and a detection result is positive when the amplifiable reagent is amplified and the testing target is amplified, and determining that the testing target is absent or at least less than the specific copy number of the amplifiable reagent and a detection result is negative when the amplifiable reagent is amplified and the testing target is not amplified, preferably includes an obtaining step of obtaining a result of amplification of the amplifiable reagent and a result of amplification of the testing target and an analyzing step of analyzing the result of amplification of the amplifiable reagent and the result of amplification of the testing target, and further includes other steps as needed.

A detection determining device of the present disclosure is a detection determining device used in detection of a testing target in a sample by amplification of the testing target and an amplifiable reagent. The amplifiable reagent is provided in a specific copy number of 200 or less. The detection determining device includes a determining unit configured to determine that the testing target is present and a detection result is positive when the amplifiable reagent is amplified and the testing target is amplified, and determine that the testing target is absent or at least less than the specific copy number of the amplifiable reagent and a detection result is negative when the amplifiable reagent is amplified and the testing target is not amplified, preferably includes an obtaining unit configured to obtain a result of amplification of the amplifiable reagent and a result of amplification of the testing target and an analyzing unit configured to analyze the result of amplification of the amplifiable reagent and the result of amplification of the testing target, and further includes other units as needed.

A detection determining program of the present disclosure is a detection determining program used in detection of a testing target in a sample by amplification of the testing target and an amplifiable reagent. The amplifiable reagent is provided in a specific copy number of 200 or less. The detection determining program preferably causes a computer to execute a process including determining that the testing target is present and a detection result is positive when the amplifiable reagent is amplified and the testing target is amplified, and determining that the testing target is absent or at least less than the specific copy number of the amplifiable reagent and a detection result is negative when the amplifiable reagent is amplified and the testing target is not amplified.

Control being performed by, for example, a control unit of the detection determining device of the present disclosure has the same meaning as the detection determining method of the present disclosure being carried out. Therefore, details of the detection determining method of the present disclosure will also be specified through description of the detection determining device of the present disclosure. Further, the detection determining program of the present disclosure realizes the detection determining device of the present disclosure with the use of, for example, computers as hardware resources. Therefore, details of the detection determining program of the present disclosure will also be specified through description of the detection determining device of the present disclosure.

The detection determining method of the present disclosure, the detection determining device of the present disclosure, and the detection determining program of the present disclosure are based on the premise that the present disclosure uses a device including wells into which an amplifiable reagent in a specific copy number is dispensed at a certain accuracy and with a coefficient of variation of higher than or equal to a certain level. Detailed description of the device will be provided below.

Detection of a testing target in a sample using the device of the present disclosure makes it possible to more securely avoid a false-negative determination in the detection of the testing target contained in the sample, particularly when the copy number of the testing target is low.

When a detection result is negative, the present disclosure ensures that the testing target, even if present, is at least less than the specific copy number of the amplifiable reagent. That is, the present disclosure ensures a “negative” determination result from a quantitative point of view of what quantity can be said to represent a state that there is almost no testing target.

In the present disclosure, “a low copy number” means that the copy number is low.

The detection determining method of the present disclosure, the detection determining device of the present disclosure, the detection determining program of the present disclosure can work more effectively for a sample containing a testing target in a low copy bumber. For example, the specific copy number of the testing target is preferably 200 or less, more preferably 100 or less, and particularly preferably 10 or less. That is, the specific copy number of the testing target of 1, 2, 3, 4, 5, 6, 7, 8, 9, or 10 is particularly preferable. As the testing target, a nucleic acid is preferable because a nucleic acid can be amplified with existing techniques.

In the following description of the detection determining method, the detection determining device, and the detection determining program of the present disclosure, a case where the testing target is a nucleic acid will be described as an example.

In the present disclosure, an amplifiable reagent is not particularly limited and may be appropriately used. In the present embodiment, a nucleic acid can be suitably used, and details will be described below. The following description employs a case of a nucleic acid.

A copy number means the number of target or specific base sequences in an amplifiable reagent contained in the well.

The target base sequence refers to a base sequence including defined base sequences in at least primer and probe regions. Specifically, a base sequence having a defined total length is also referred to as specific base sequence.

A specific copy number refers to the aforementioned copy number that specifies the number of target base sequences at accuracy of a certain level or higher.

This means that the specific copy number is known as the number of target base sequences actually contained in a well. That is, the specific copy number in the present disclosure is more accurate or reliable as a number than a predetermined copy number (calculated estimated value) obtained according to existing serial dilution methods, and is a controlled value that has no dependency on a Poisson distribution even if the value is within a low copy number region of 1,000 or lower in particular. When it is said that the specific copy number is a controlled value, it is preferable that a coefficient of variation CV expressing uncertainty roughly satisfy either CV<1/√x with respect to an average copy number x or CV≤20%. Hence, use of a device including wells in which a target base sequence is contained in the specific copy number makes it possible to perform qualitative or quantitative testing of samples containing the target base sequence more accurately than ever.

When the number of target base sequences and the number of nucleic acid molecules including the sequence coincide with each other, “copy number” and “number of molecules” may be associated with each other.

Specifically, for example, in the case of norovirus, when the number of viruses is 1, the number of nucleic acid molecules is 1 and the copy number is 1. In the case of yeast at a GI phase, when the number of yeast cells is 1, the number of nucleic acid molecules (the number of same chromosomes) is 1 and the copy number is 1. In the case of human cell at a G0/GI phase, when the number of human cells is 1, the number of nucleic acid molecules (the number of same chromosomes) is 2 and the copy number is 2.

Further, in the case of yeast at a GI phase having the target base sequence introduced at two positions, when the number of yeast cells is 1, the number of nucleic acid molecules (the number of same chromosomes) is 1 and the copy number is 2.

In the present disclosure, a specific copy number of the amplifiable reagent may be referred to as predetermined number or absolute number of the amplifiable reagent. The copy number of the amplifiable reagent is preferably 200 or less, more preferably 100 or less, and particularly preferably 10 or less. That is, the copy number of the amplifiable reagent of 1, 2, 3, 4, 5, 6, 7, 8, 9, or 10 is particularly preferable.

<Determining Step and Determining Unit>

The determining step is a step of using an amplifiable reagent in a specific copy number, determining that a testing target is present and a detection result is positive when the amplifiable reagent is amplified and the testing target is amplified, and determining that the testing target is absent or at least less than the specific copy number of the amplifiable reagent and a detection result is negative when the amplifiable reagent is amplified and the testing target is not amplified, and is performed by the determining unit.

When the copy number of reference DNA used as control is not specified as in PTL 1 or 2 mentioned above, for example, determination about detection of the testing target made based on the result of amplification of the testing target and the result of amplification of the amplifiable reagent will result as presented in Table 1 below.

TABLE 1

| | | Amplifiable reagent, the copy number of | |
|---|---|---|---|
| | | which is not specified | |
| | | + | − |
| Testing | + | Positive (highly probable) | Reconsideration of PCR |
| target in | − | Negative or false-negative | reaction system and copy |
| sample | | (impossible to specify | number of amplifiable |
| | | whether negative or false- | reagent is needed |
| | | negative) | |

As presented in Table 1, amplification reaction results include four patterns, namely (1) a case where amplification is observed in both of the nucleic acid in the sample and the reference nucleic acid serving as the amplifiable reagent, (2) a case where amplification is observed in the reference nucleic acid serving as the amplifiable reagent, but amplification is not observed in the nucleic acid in the sample, (3) a case where amplification is observed in the nucleic acid in the sample, but amplification is not observed in the reference nucleic acid serving as the amplifiable reagent, and (4) a case where amplification is observed in neither the nucleic acid in the sample nor the reference nucleic acid serving as the amplifiable reagent.

When the copy number of the amplifiable reagent is not specified as in Table 1, the results (1) to (4) described above can be determined as follows.

In the case of (1), it is possible to confirm that the experiment by PCR reaction has been successful because amplification is observed in the reference nucleic acid serving as the amplifiable reagent. Further, it is possible to confirm that the testing target nucleic acid is present in the sample because amplification is observed in the nucleic acid in the sample.

In the case of (2), it is possible to confirm that the experiment by PCR reaction has been successful because amplification is observed in the reference nucleic acid serving as the amplifiable reagent. However, because amplification is not observed in the nucleic acid in the sample, it is generally determined that the testing target nucleic acid is absent in the sample. However, because the copy number of the reference nucleic acid serving as the amplifiable reagent is not specified, it is impossible to specify which of the following cases is pertinent, namely a case where the testing target nucleic acid is truly absent in the sample (negative) and a case where the testing target nucleic acid is present in the sample but in a trace amount, and cannot be experimentally identified and was erroneously determined as negative (false-negative). Particularly, when the copy number of the nucleic acid is a low copy number, the determination of whether negative or false-negative is more difficult.

In the case of (3) and (4), because amplification is not observed in the reference nucleic acid serving as the amplifiable reagent, for example, it is estimated that the PCR reaction has not progressed due to some causes (for example, reaction temperature conditions, preparation of the amplifiable reagent, a thermal cycler, and settings of the real-time PCR device), or that the copy number of the amplifiable reagent is insufficient with respect to the limit of detection, and it is determined that “reconsideration of the PCR reaction system and the copy number of the amplifiable reagent is needed”. When the copy number of the amplifiable reagent is not specified, the copy number has a large variation, and the probability that the copy number is higher than or equal to the limit of detection is low. This inevitably increases the frequency that the test results of (3) and (4) will be obtained. Therefore, when the copy number of the amplifiable reagent is not specified, there is a need for performing a test at a copy number that is twice or three times higher than the limit of detection.

On the other hand, when the copy number of the reference nucleic acid serving as the amplifiable reagent is specified as in the present disclosure, i.e., when the copy number is a specific copy number, for example, determination about detection of the testing target made based on the result of amplification of the testing target and the result of amplification of the amplifiable reagent will result as presented in Table 2 below.

TABLE 2

| | | Amplifiable reagent in specific copy number | |
|---|---|---|---|
| | | + | − |
| Testing | + | Positive (definite) | Reconsideration of PCR |
| target in | − | Negative or at least less than | reaction system and copy |
| sample | | specific copy number | number of amplifiable |
| | | | reagent is needed |

When the copy number of the reference nucleic acid serving as the amplifiable reagent is specified as presented in Table 2, the results (1) to (4) described above can be determined as follows.

In the case of (1), it is possible to determine definitely that the experiment by PCR reaction has been successful because amplification is observed in the reference nucleic acid serving as the amplifiable reagent. Further, it is possible to determine definitely that the testing target nucleic acid is present in the sample because amplification is observed in the nucleic acid in the sample. Even though the copy number of the nucleic acid is a low copy number, the “positive” determination result can be ensured.

In the case of (2), it is possible to confirm that the experiment by PCR reaction has been successful because amplification is observed in the reference nucleic acid serving as the amplifiable reagent. However, because amplification is not observed in the nucleic acid in the sample, it is possible to determine definitely that the nucleic acid in the sample is at least less than the specific copy number of the amplifiable reagent. That is, it is possible to determine that the testing target nucleic acid is absent in the sample or at least less than the specific copy number of the amplifiable reagent, and that the detection result is “negative” meaning that the testing target is absent, or “at least less than the specific copy number”. In the case of (2), it is impossible to specify whether negative or false-negative according to Table 1, whereas it is possible to conclude that the result is “negative” or “at least less than the specific copy number” as described above according to Table 2 of the present disclosure because the copy number of the reference nucleic acid serving as the amplifiable reagent is specified.

The present disclosure makes it possible to more securely exclude false-negative and improve the accuracy for negative determination. The present disclosure can reduce false-negative and ensure a “negative” determination result based on the reasoning that the testing target nucleic acid is at least less than the specific copy number of the amplifiable reagent.

In the case of (3) and (4), because amplification is not observed in the reference nucleic acid serving as the amplifiable reagent, for example, it is estimated that the PCR reaction has not progressed due to some causes (for example, reaction temperature conditions, preparation of the amplifiable reagent, a thermal cycler, and settings of the real-time PCR device), or that the copy number of the amplifiable reagent is insufficient with respect to the limit of detection, and it is determined that “reconsideration of the PCR reaction system and the copy number of the amplifiable reagent is needed”.

In the detection determining method of the present disclosure, when there is a limit of detection by the copy number, it is preferable that the limit of detection of the testing target nucleic acid be comparable to the limit of detection of the nucleic acid serving as the amplifiable reagent.

This makes it possible to regard a limit of detection obtained based on a result of amplification of the reference nucleic acid serving as the amplifiable reagent as a limit of detection of the testing target nucleic acid.

In the detection determining method of the present disclosure, it is preferable to perform amplification reactions of the testing target nucleic acid and the nucleic acid serving as the amplifiable reagent, using a device described below.

The device includes at least one sample filling well to be filled with a sample. The sample filling well further includes an amplifiable reagent in a specific copy number. The specific copy number of the amplifiable reagent is a specific natural number of 200 or less.

That is, it is more preferable to fill the amplifiable reagent in the sample filling well to be filled with a sample and perform amplification reactions of the testing target and the amplifiable reagent in the same sample filling well, using the device. By performing amplification reactions of the testing target and the amplifiable reagent in the same well, it is possible to suppress variations of reaction conditions and increase reliability of the results of amplification.

In the detection determining method of the present disclosure, it is preferable to perform amplification reactions of the testing target and the amplifiable reagent, using nucleic acids having different base sequences from each other as the testing target and the amplifiable reagent.

Further, in the detection determining method of the present disclosure, it is preferable to perform amplification reactions, by filling a positive control having the same base sequence as the testing target base sequence in a certain amount in a different well from the sample filling well. Here, the certain amount needs at least to be a sufficiently detectable amount.

With a positive control filled in a different well, it is ensured more reliably that the determinations in the case of (1) and (2) in Table 2 are correct, provided that amplification of the positive control is observed.

The device used in the detection determining method of the present disclosure will be described in more detail below.

<Detection Result Obtaining Step and Detection Result Obtaining Unit>

A detection result obtaining step is a step of obtaining a result of amplification of the nucleic acid serving as the amplifiable reagent and a result of amplification of the testing target nucleic acid, and is performed by a detection result obtaining unit.

A detection result obtaining unit 131 is configured to obtain a result of amplification of the nucleic acid serving as the amplifiable reagent and a result of amplification of the testing target nucleic acid obtained from PCR reactions. The data of the obtained results of amplification is stored in a detection result database 141.

<Detection Result Analyzing Step and Detection Result Analyzing Unit>

A detection result analyzing step is a step of analyzing the obtained result of amplification of the nucleic acid serving as the amplifiable reagent and the obtained result of amplification of the testing target nucleic acid, and is performed by a detection result amplifying unit.

A detection result analyzing unit 132 is configured to obtain the data of the results of amplification stored in the detection result database 141, and based on the data, analyze whether amplification is observed in the nucleic acid serving as the amplifiable reagent and whether amplification is observed in the testing target nucleic acid.

The procedure of a detection determining program of the present disclosure can be executed using a computer including a control unit constituting a detection determining device.

The hardware configuration and the functional configuration of the detection determining device will be described below.

<Hardware Configuration of Detection Determining Device>

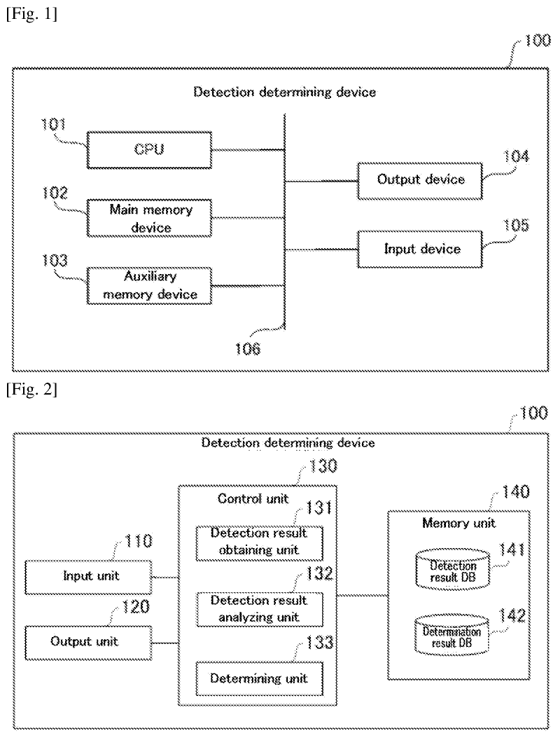

FIG. 1 is a block diagram illustrating an example of the hardware configuration of a detection determining device 100.

As illustrated in FIG. 1, the detection determining device 100 includes units such as a CPU (Central Processing Unit) 101, a main memory device 102, an auxiliary memory device 103, an output device 104, and an input device 105. These units are coupled to one another through a bus 106.

The CPU 101 is a processing device configured to execute various controls and operations. The CPU 101 realizes various functions by executing OS (Operating System) and programs stored in, for example, the main memory device 102. That is, in the present example, the CPU 101 functions as a control unit 130 of the detection determining device 100 by executing the detection determining program.

The CPU 101 also controls the operation of the entire detection determining device 100. In the present example, the CPU 101 is used as the device configured to control the operation of the entire detection determining device 100. However, this is nonlimiting. For example, FPGA (Field Programmable Gate Array) may be used.

The detection determining program and various databases need not indispensably be stored in, for example, the main memory device 102 and the auxiliary memory device 103. The detection determining program and various databases may be stored in, for example, another information processing device that is coupled to the detection determining device 100 through, for example, the Internet, a LAN (Local Area Network), and a WAN (Wide Area Network). The detection determining device 100 may receive the detection determining program and various databases from such another information processing device and execute the program and databases.

The main memory device 102 is configured to store various programs and store, for example, data needed for execution of the various programs.

The main memory device 102 includes a ROM (Read Only Memory) and a RAM (Random Access Memory) that are not illustrated.

The ROM is configured to store various programs such as BIOS (Basic Input/Output System).

The RAM functions as a work area to be developed when the various programs stored in the ROM are executed by the CPU 101. The RAM is not particularly limited and may be appropriately selected depending on the intended purpose. Examples of the RAM include a DRAM (Dynamic Random Access Memory) and a SRAM (Static Random Access Memory).

The auxiliary memory device 103 is not particularly limited and may be appropriately selected depending on the intended purpose so long as the auxiliary memory device 103 can store various information. Examples of the auxiliary memory device 103 include portable memory devices such as a CD (Compact Disc) drive, a DVD (Digital Versatile Disc) drive, and a BD (Blue-ray (registered trademark) Disc) drive.

For example, a display or a speaker can be used as the output device 104. The display is not particularly limited and a known display can be appropriately used. Examples of the display include a liquid crystal display and an organic EL display.

The input device 105 is not particularly limited and a known input device can be appropriately used so long as the input device can receive various requests to the detection determining device 100. Examples of the input device include a keyboard, a mouse, and a touch panel.

The hardware configuration as described above can realize the process functions of the detection determining device 100.

<Functional Configuration of Detection Determining Device>

FIG. 2 is a diagram illustrating an example of the functional configuration of the detection determining device 10.

As illustrated in FIG. 2, the detection determining device 100 includes an input unit 110, an output unit 120, the control unit 130, and a memory unit 140.

The control unit 130 includes the detection result obtaining unit 131, the detection result analyzing unit 132, and a determining unit 133. The control unit 130 is configured to control the entire detection determining device 100.

The memory unit 140 includes the detection result database 141 and a determination result database 142. Hereinafter, “database” may be referred to as “DB”.

The detection result obtaining unit 131 is configured to obtain a result of amplification of a nucleic acid serving as an amplifiable reagent and a result of amplification of a testing target nucleic acid obtained from PCR reactions. The control unit 130 is configured to store data of the obtained results of amplification in the detection result DB 141.

The detection result analyzing unit 132 is configured to analyze the result of amplification of the nucleic acid serving as the amplifiable reagent and the result of amplification of the testing target nucleic acid, using the data of the results of amplification stored in the detection result DB 141 of the memory unit 140.

The determining unit 133 is configured to determine “positive” and “negative” when the classifications described below are applicable, based on the results of the analyses of the detection result analyzing unit 132.

(1) When the amplifiable reagent is amplified and the testing target is amplified, it is determined that the testing target is present and the detection result is positive.

(2) When the amplifiable reagent is amplified and the testing target is not amplified, it is determined that the testing target is absent or at least less than the specific copy number of the amplifiable reagent and the detection result is negative.

In addition to the determinations of (1) and (2) above, the determining unit 133 may make a determination of, for example, failure of experiment when the cases of (3) and (4) in Table 2 are applicable.

The control unit 130 is configured to store the determination result of the determining unit 133 in the determination result DB 142.

Next, the process procedure of the detection determining program of the present disclosure will be described. FIG. 3 is a flowchart illustrating an example of the process procedure of the detection determining program by the control unit 130 of the detection determining device 100.

In the steps S101, the detection result obtaining unit 131 of the control unit 130 of the detection determining device 100 obtains a result of amplification of a nucleic acid serving as an amplifiable reagent and a result of amplification of a testing target nucleic acid obtained from PCR reactions, and moves the flow to the step S102. In the step S101, the control unit 130 stores the data of the results of amplification obtained by the detection result obtaining unit 131 in the detection result DB 141 of the memory unit 140.

In the step S102, the detection result analyzing unit 132 of the control unit 130 of the detection determining device 100 obtains the data of the results of amplification stored in the detection result DB 141. Then, the detection result analyzing unit 132 analyzes the respective results as to whether amplification is observed in the nucleic acid serving as the amplifiable reagent and whether amplification is observed in the testing target nucleic acid, and moves the flow to the step S103.

In the step S103, the determining unit 133 of the control unit 130 of the detection determining device 100 moves the flow to the step S104 when amplification is observed in the nucleic acid serving as the amplifiable reagent, based on the result of the analysis by the detection result analyzing unit 132. On the other hand, the determining unit 133 moves the flow to the step S107 when amplification is not observed in the nucleic acid serving as the amplifiable reagent.

In the step S104, the determining unit 133 moves the flow to the step S105 when amplification is observed in the testing target nucleic acid, based on the result of the analysis by the detection result analyzing unit 132. On the other hand, the determining unit 133 moves the flow to step S106 when amplification is not observed in the testing target nucleic acid.

In the step S105, the determining unit 133 determines that the testing target is present and the detection result is positive, based on the results that the nucleic acid serving as the amplifiable reagent is amplified and that the testing target nucleic acid is amplified, and moves the flow to the step S110.

In the step S106, the determining unit 133 determines that the testing target is absent or at least less than the specific copy number of the amplifiable reagent and the detection result is negative, based on the results that the nucleic acid serving as the amplifiable reagent is amplified and that the testing target nucleic acid is not amplified, and moves the flow to the step S110.

In the step S107, the determining unit 133 moves the flow to the step S108 when amplification is observed in the testing target nucleic acid, based on the result of the analysis by the detection result analyzing unit 132. On the other hand, the determining unit 133 moves the flow to step S109 when amplification is not observed in the testing target nucleic acid.

In the step S108, the determining unit 133 determines that reconsideration of the PCR reaction system and the copy number of the amplifiable reagent is needed, based on the results that the nucleic acid serving as the amplifiable reagent is not amplified and that the testing target nucleic acid is amplified, and moves the flow to the step S110.

In the step S109, the determining unit 133 determines that reconsideration of the PCR reaction system and the copy number of the amplifiable reagent is needed, based on the results that the nucleic acid serving as the amplifiable reagent is not amplified and that the testing target nucleic acid is not amplified, and moves the flow to the step S110. In the step S110, the control unit 130 stores the determination result made by the determining unit 133 in the determination result DB 142 of the memory unit 140 and terminates the flow.

In the present disclosure, it is at least needed to make the determination in the step S105 or the step S106, and a mode in which the flow is terminated without moving to the step S107 is possible when amplification is not observed in the nucleic acid serving as the amplifiable reagent.

The device used in the detection determining program of the present disclosure, the detection determining method of the present disclosure, and the detection determining device of the present disclosure will be described below.

In the present specification, a device including an amplifiable reagent will be referred to as “device”. A device including no amplifiable reagent will be referred to as “plate”.

(Device)

A device of the present disclosure includes at least one sample filling well to be filled with a sample. The sample filling well further includes an amplifiable reagent in a specific copy number. The specific copy number of the amplifiable reagent is 200 or less. The device further includes other members as needed.

According to the device of the present disclosure, the amplifiable reagent is filled in the sample filling well to be filled with a sample. Therefore, it is possible to perform amplification reactions of the testing target and the amplifiable reagent in the same well. This makes it possible to suppress variations of reaction conditions and increase reliability of the results of amplification.

As the amplifiable reagent, a nucleic acid can be suitably used. Nucleic acid will be described in detail below.

A copy number means the number of target or specific base sequences in an amplifiable reagent contained in the well.

The target base sequence refers to a base sequence including defined base sequences in at least primer and probe regions. Specifically, a base sequence having a defined total length is also referred to as specific base sequence.

A specific copy number refers to the aforementioned copy number that specifies the number of target base sequences at accuracy of a certain level or higher.

This means that the specific copy number is known as the number of target base sequences actually contained in a well. That is, the specific copy number in the present disclosure is more accurate or reliable as a number than a predetermined copy number (calculated estimated value) obtained according to existing serial dilution methods, and is a controlled value that has no dependency on a Poisson distribution even if the value is within a low copy number region of 1,000 or lower in particular. When it is said that the specific copy number is a controlled value, it is preferable that a coefficient of variation CV expressing uncertainty roughly satisfy either CV<1/√x with respect to an average copy number x or CV≤20%. Hence, use of a device including wells in which a target base sequence is contained in the specific copy number makes it possible to perform qualitative or quantitative testing of samples containing the target base sequence more accurately than ever.

When the number of target base sequences and the number of nucleic acid molecules including the sequence coincide with each other, “copy number” and “number of molecules” may be associated with each other.

Specifically, for example, in the case of norovirus, when the number of viruses is 1, the number of nucleic acid molecules is 1 and the specific copy number is 1. In the case of yeast at a GI phase, when the number of yeast cells is 1, the number of nucleic acid molecules (the number of same chromosomes) is 1 and the specific copy number is 1. In the case of human cell at a G0/GI phase, when the number of human cells is 1, the number of nucleic acid molecules (the number of same chromosomes) is 2 and the copy number is 2.

Further, in the case of yeast at a GI phase having the target base sequence introduced at two positions, when the number of yeast cells is 1, the number of nucleic acid molecules (the number of same chromosomes) is 1 and the copy number is 2.

In the present disclosure, a specific copy predetermined number of the amplifiable reagent may also be referred to as specific copy number or absolute number of the amplifiable reagent.

The copy number of the amplifiable reagent is preferably 200 or less, more preferably 100 or less, and particularly preferably 10 or less. That is, the copy number of the amplifiable reagent of 1, 2, 3, 4, 5, 6, 7, 8, 9, or 10 is particularly preferable.

The specific copy number of the amplifiable reagent may include two or more different integers.

Examples of the combination of specific copy numbers of the amplifiable reagent include a combination of 1, 2, 3, 4, 5, 6, 7, 8, 9, and 10, a combination of 1, 3, 5, 7, and 9, and a combination of 2, 4, 6, 8, and 10.

For example, the combination of specific copy numbers of the amplifiable reagent may be a combination of the following four levels: 1, 10, 100, and 1,000. By using the device of the present disclosure with a combination of a plurality of different specific copy numbers, it is possible to generate a calibration curve.

It is preferable that a coefficient of variation of the sample filling well be lower than or equal to a coefficient of variation CV of the specific copy number of the amplifiable reagent.

It is preferable that the sample filling well include information on the specific copy number of the amplifiable reagent and uncertainty based on the specific copy number of the amplifiable reagent.

The coefficient of variation CV and the information on the uncertainty will be described below.

While being dissolved in solvent molecules, solute molecules of, for example, a nucleic acid sample migrate through the solvent molecules due to thermal fluctuation. In this case, the distribution state of the molecules is generally said to conform to a Poisson distribution. This indicates that the number of molecules in the solution filled in a container has a distribution, i.e., a variation (coefficient of variation), regardless of with what level of accuracy the solution having a prescribed concentration is weighed out and filled in the container. When the same base sequence is not to be introduced in a plural number into one molecule, “a number of molecules” may be used in the same meaning as “a copy number”.

Here, the coefficient of variation means a relative value of the variation in the number of cells filled in each concave, where the variation occurs when cells are filled in the concave. That is, the coefficient of variation means the filling accuracy in terms of the number of cells (or amplifiable reagents) filled in the concave. The coefficient of variation is a value obtained by dividing standard deviation σ by an average value x. Here, the coefficient of variation CV is assumed to be a value obtained by dividing standard deviation σ by an average copy number (average number of copies filled) x. In this case, a relational expression represented by Formula 1 below is established.

$$\begin{matrix}[ Math . 1 ] & \\ CV = \frac{σ}{x} & Formula 1\end{matrix}$$

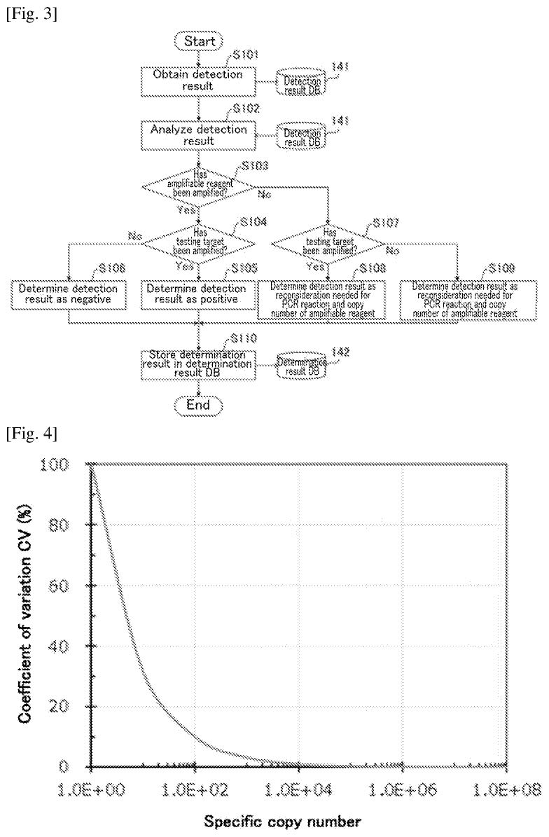

Generally, cells (or amplifiable reagents) have a random distribution state of a Poisson distribution in a dispersion liquid. Therefore, in a random distribution state by a serial dilution method, i.e., of a Poisson distribution, standard deviation σ can be regarded as satisfying a relational expression represented by Formula 2 below with an average copy number x. Hence, in the case where a dispersion liquid of cells (or amplifiable reagents) is diluted by a serial dilution method, when coefficients of variation CV (CV values) for average copy numbers x are calculated according to Formula 3 below derived from Formula 1 above and Formula 2 based on the standard deviation σ and the average copy numbers x, the results are as presented in Table 3 and FIG. 4.

$$\begin{matrix}[ Math . 2 ] & \\ σ = \sqrt{x} & Formula 2 \\ [ Math . 3 ] & \\ CV = \frac{1}{\sqrt{x}} & Formula 3\end{matrix}$$

TABLE 3

| | Coefficient of variation |
|---|---|
| Average copy number x | CV |
| 1.00E+00 | 100.00% |
| 1.00E+01 | 31.62% |
| 1.00E+02 | 10.00% |
| 1.00E+03 | 3.16% |
| 1.00E+04 | 1.00% |
| 1.00E+05 | 0.32% |
| 1.00E+06 | 0.10% |
| 1.00E+07 | 0.03% |
| 1.00E+08 | 0.01% |

From the results of Table 3 and FIG. 4, it can be understood that when a well is to be filled with, for example, a copy number of 100 of nucleic acid samples according to a serial dilution method, the final copy number of nucleic acid samples to be filled in the reaction solution has a coefficient of variation (CV) of at least 10%, even when other accuracies are ignored.

The coefficient of variation is a value obtained by dividing standard deviation σ by an average copy number x, and a term “CV value” is used as abbreviation. The coefficient of variation CV for a copy number having variation according to a Poisson distribution can be obtained from FIG. 4.

When the copy number of the amplifiable reagent is 200 or less, it is preferable that the coefficient of variation CV of the sample filling well and an average specific copy number x of the amplifiable reagent satisfy the following relationship: CV<1/√x.

In the case where the amplifiable reagent is provided in a specific copy number in the sample filling well, it is preferable that the well include information on uncertainty based on the specific copy number.

Uncertainty is defined in ISO/IEC Guide 99:2007 [International Vocabulary of Metrology-Basics and general concepts and related terms (VIM)] as “a parameter that characterizes measurement result-incidental variation or dispersion of values rationally linkable to the measured quantity”. Here, “values rationally linkable to the measured quantity” means candidates for the true value of the measured quantity. That is, uncertainty means information on the variation of the results of measurement due to operations and devices involved in production of a measurement target. With a greater uncertainty, a greater variation is predicted in the results of measurement.

For example, the uncertainty may be standard deviation obtained from the results of measurement, or a half value of a reliability level, which is expressed as a numerical range in which the true value is contained at a predetermined probability or higher. The uncertainty may be calculated according to the methods based on, for example, Guide to the Expression of Uncertainty in Measurement (GUM:ISO/IEC Guide 98-3), and Japan Accreditation Board Note 10, Guideline on Uncertainty in Measurement in Test. As the method for calculating the uncertainty, for example, there are two types of applicable methods: a type-A evaluation method using, for example, statistics of the measured values, and a type-B evaluation method using information on uncertainty obtained from, for example, calibration certificate, manufacturer's specification, and information open to the public.

All uncertainties due to factors such as operations and measurement can be expressed by the same reliability level, by conversion of the uncertainties to standard uncertainty. Standard uncertainty indicates variation in the average value of measured values.

In an example method for calculating the uncertainty, for example, factors that may cause uncertainties are extracted, and uncertainties (standard deviations) due to the respective factors are calculated. Then, the calculated uncertainties due to the respective factors are synthesized according to the sum-of-squares method, to calculate a synthesized standard uncertainty. In the calculation of the synthesized standard uncertainty, the sum-of-squares method is used. Therefore, a factor that causes a sufficiently small uncertainty can be ignored, among the factors that cause uncertainties. As the uncertainty, a coefficient of variation (CV) obtained by dividing the synthesized standard uncertainty by an expected value may be used.

It is preferable to calculate the uncertainty to be associated with each well appropriately by the filling method described above or a dilution series producing method.

As the information on the uncertainty of the specific copy number of the amplifiable reagent, all factors that are involved in production of the device can be taken into consideration. Examples include information on the factors presented below.

There are some conceivable factors that cause uncertainties. For example, in a production process of introducing the intended amplifiable reagent into cells and dispensing the cells while counting the number of cells, examples of the conceivable factors include the number of amplifiable reagents in a cell (for example, the cell cycl of the cell), the unit configured to locate the cells in the device (including any outcomes of operations of an inkjet device or each section of the device, such as operation timings of the device, and the number of cells included in a liquid droplet when the cell suspension is formed into the form of a liquid droplet), the frequency at which cells are located at appropriate positions of the device (for example, the number of cells located in a well), and contamination due to destruction of cells in a cell suspension and consequent mixing of the amplifiable reagent into the cell suspension (hereinafter may also be described as mixing of contaminants).

As presented in Examples below, the coefficient of variation CV can be obtained by calculating an average copy number of the amplifiable reagent and uncertainty based on experiment results, and dividing the uncertainty (standard deviation (5) by the average copy number x.

It is preferable that a sample filling well contain at least any one of a primer and an amplifying reagent.

A primer is a synthetic oligonucleotide having a complementary base sequence that includes from 18 through 30 bases and is specific to a template DNA of a polymerase chain reaction (PCR). A pair of primers, namely a forward primer and a reverse primer, are set at two positions in a manner to sandwich the region to be amplified.

Examples of the amplifying reagent for a polymerase chain reaction (PCR) include enzymes such as DNA polymerase, matrices such as the four kinds of bases (dGTP, dCTP, dATP, and dTTP), Mg²⁺ (2 mM magnesium chloride), and a buffer for maintaining the optimum pH (pH of from 7.5 through 9.5).

The base sequence of the amplifiable reagent may be different from the base sequence of the testing target. This is a preferable mode for performing amplification reactions of the testing target and the amplifiable reagent in the same well.

Because the base sequence of the amplifiable reagent and the base sequence of the testing target are different from each other, a preferable mode is a mode in which a pair of primers for amplifying the testing target and a pair of primers for amplifying the amplifiable reagent are introduced into the sample filling well.

The device of the present disclosure has a mode in which a positive control having the same base sequence as the base sequence of the testing target is filled in a certain amount in a different well from the sample filling well. Here, the certain amount needs at least to be a sufficiently detectable amount. With a positive control filled in a different well, it can be ensured more reliably that the determinations in the case of (1) and (2) in Table 2 are correct, provided that amplification of the positive control is observed.

The device of the present disclosure includes at least one sample filling well, preferably includes an identifier unit and a base material, and further includes other members as needed.

In the present disclosure, a well to be filled with a positive control may be provided in a plate in addition to a well used for sample filling. In the following, general description of wells including the sample filling well will be provided.

<Wells>

For example, the shape, the number, the volume, the material, and the color of the well are not particularly limited and may be appropriately selected depending on the intended purpose.

The shape of the well is not particularly limited and may be appropriately selected depending on the intended purpose so long as, for example, a nucleic acid can be placed in the well. Examples of the shape of the well include: concaves such as a flat bottom, a round bottom, a U bottom, and a V bottom; and sections on a substrate.

The number of wells is at least one, preferably a plural number of 2 or greater, more preferably 5 or greater, and yet more preferably 50 or greater.

Examples of a one-well product include a PCR tube.

Preferable examples of a two or more-well product include a multi-well plate.

Examples of the multi-well plate include a 24-well, 48-well, 96-well, 384-well, or 1,536-well plate.

The volume of the well is not particularly limited, may be appropriately selected depending on the intended purpose, and is preferably 10 microliters or greater but 1,000 microliters or less in consideration of the amount of a sample used in a common nucleic acid testing device.

The material of the well is not particularly limited and may be appropriately selected depending on the intended purpose. Examples of the material of the well include polystyrene, polypropylene, polyethylene, fluororesins, acrylic resins, polycarbonate, polyurethane, polyvinyl chloride, and polyethylene terephthalate.

Examples of the color of the well include transparent colors, semi-transparent colors, chromatic colors, and complete light-shielding colors.

Wettability of the well is not particularly limited and may be appropriately selected depending on the intended purpose. The wettability of the well is preferably water repellency. When the wettability of the well is water repellency, adsorption of the amplifiable reagent to the internal wall of the well can be reduced. Further, when the wettability of the well is water repellency, the amplifiable reagent, a primer, and an amplifying reagent in the well can be moved in a state of a solution.

The method for imparting water repellency to the internal wall of the well is not particularly limited and may be appropriately selected depending on the intended purpose. Examples of the method include a method of forming a fluororesin coating film, a fluorine plasma treatment, and an embossing treatment. Particularly, by applying a water repellency imparting treatment that imparts a contact angle of 100 degrees or greater, it is possible to suppress reduction of the amplifiable reagent due to spill of the liquid and suppress increase of uncertainty (or coefficient of variation).

<Base Material>

The device is preferably a plate-shaped device obtained by providing a well in a base material, but may be linking-type well tubes such as 8-series tubes.

For example, the material, the shape, the size, and the structure of the base material are not particularly limited and may be appropriately selected depending on the intended purpose.

The material of the base material is not particularly limited and may be appropriately selected depending on the intended purpose. Examples of the material of the base material include semiconductors, ceramics, metals, glass, quartz glass, and plastics. Among these materials, plastics are preferable.

Examples of the plastics include polystyrene, polypropylene, polyethylene, fluororesins, acrylic resins, polycarbonate, polyurethane, polyvinyl chloride, and polyethylene terephthalate.

The shape of the base material is not particularly limited and may be appropriately selected depending on the intended purpose. For example, board shapes and plate shapes are preferable.

The structure of the base material is not particularly limited, may be appropriately selected depending on the intended purpose, and may be, for example, a single-layer structure or a multilayered structure.

<Identifier Unit>

It is preferable that the device include an identifier unit that enables identifying information on a coefficient of variation CV and information on uncertainty for a specific copy number of the amplifiable reagent in a sample filling well.

The identifier unit is not particularly limited and may be appropriately selected depending on the intended purpose. Examples of the identifier unit include a memory, an IC chip, a barcode, a QR code (registered trademark), a Radio Frequency Identifier (hereinafter may also be referred to as “RFID”), color coding, and printing.

The position at which the identifier unit is provided and the number of identifier units are not particularly limited and may be appropriately selected depending on the intended purpose.

Example of the information to be stored in the identifier unit include not only information on a presence probability at which the amplifiable reagent in a specific copy number is present in the specific copy number in a well, but also results of analyses (for example, activity value and emission intensity), the number of amplifiable reagents (for example, the number of cells), whether cells are alive or dead, the copy number of specific base sequences, which of a plurality of wells is filled with the amplifiable reagent, the kind of the amplifiable reagent, the measurement date and time, and the name of the person in charge of measurement.

The information stored in the identifier unit can be read with various kinds of reading units. For example, when the identifier unit is a barcode, a barcode reader is used as the reading unit.

The method for writing information in the identifier unit is not particularly limited and may be appropriately selected depending on the intended purpose. Examples of the method include manual input, a method of directly writing data through a liquid droplet forming device configured to count the number of amplifiable reagents during dispensing of the amplifiable reagents into the wells, transfer of data stored in a server, and transfer of data stored in a cloud system.

<Other Members>

The other members are not particularly limited and may be appropriately selected depending on the intended purpose. Examples of the other members include a sealing member.

—Sealing Member—

It is preferable that the device include a sealing member in order to prevent mixing of foreign matters into the wells and outflow of the filled materials.

It is preferable that the sealing member be configured to be capable of sealing at least one well and separable at a perforation in order to be capable of sealing or opening each one of the wells individually.

The shape of the sealing member is preferably a cap shape matching the inner diameter of a well, or a film shape for covering the well opening.

Examples of the material of the sealing member include polyolefin resins, polyester resins, polystyrene resins, and polyamide resins.

It is preferable that the sealing member have a film shape that can seal all wells at a time. It is also preferable that the sealing member be configured to have different adhesive strengths for wells that need to be reopened and wells that need not, in order that the user can reduce improper use.

It is preferable that the amplifiable reagent be a nucleic acid. It is preferable that the nucleic acid be incorporated into the nucleus of a cell.

—Nucleic Acid—

A nucleic acid means a polymeric organic compound in which a nitrogen-containing base derived from purine or pyrimidine, sugar, and phosphoric acid are bonded with one another regularly. Examples of the nucleic acid also include a fragment of a nucleic acid or an analog of a nucleic acid or of a fragment of a nucleic acid.

The nucleic acid is not particularly limited and may be appropriately selected depending on the intended purpose. Examples of the nucleic acid include DNA, RNA, and cDNA.

The nucleic acid or nucleic acid fragment may be a natural product obtained from a living thing, or a processed product of the natural product, or a product produced by utilizing a genetic recombination technique, or a chemically synthesized artificially synthesized nucleic acid molecule. One of these nucleic acids may be used alone or two or more of these nucleic acids may be used in combination. With an artificially synthesized nucleic acid molecule, it is possible to suppress impurities and set the molecular weight to a low level. This makes it possible to improve the initial reaction efficiency.

An artificially synthesized nucleic acid means an artificially synthesized nucleic acid produced to have the same constituent components (base, deoxyribose, and phosphoric acid) as naturally existent DNA or RNA. Examples of the artificially synthesized nucleic acid include not only a nucleic acid having a base sequence coding a protein, but also a nucleic acid having an arbitrary base sequence.

Examples of the analog of a nucleic acid or a nucleic acid fragment include a nucleic acid or a nucleic acid fragment bonded with a non-nucleic acid component, a nucleic acid or a nucleic acid fragment labeled with a labeling agent such as a fluorescent dye or an isotope (e.g., a primer or a probe labeled with a fluorescent dye or a radioisotope), and an artificial nucleic acid, which is a nucleic acid or a nucleic acid fragment in which the chemical structure of some of the constituent nucleotides is changed (e.g., PNA, BNA, and LNA).

The form of the nucleic acid is not particularly limited and may be appropriately selected depending on the intended purpose. Examples of the form of the nucleic acid include double-strand nucleic acid, single-strand nucleic acid, and partially double-strand or single-strand nucleic acid. Cyclic or straight-chain plasmids can also be used.

The nucleic acid may be modified or mutated.

It is preferable that the nucleic acid have a target base sequence.

The target base sequence is not particularly limited and may be appropriately selected depending on the intended purpose. Examples of the target base sequence include base sequences used for infectious disease testing, naturally non-existent non-natural base sequences, animal cell-derived base sequences, plant cell-derived base sequences, fungal cell-derived base sequences, bacterium-derived base sequences, and virus-derived base sequences. One of these base sequences may be used alone or two or more of these base sequences may be used in combination.

When using a non-natural base sequence, the target base sequence preferably has a GC content of 30% or higher but 70% or lower, and preferably has a constant GC content (for example, see SEQ ID NO. 1).

The base length of the target base sequence is not particularly limited, may be appropriately selected depending on the intended purpose, and may be, for example, a base length of 20 base pairs (or mer) or greater but 10,000 base pairs (or mer).

When using a base sequence used for infectious disease testing, the base sequence is not particularly limited and may be appropriately selected depending on the intended purpose so long as the base sequence includes a base sequence specific to the intended infectious disease. It is preferable that the base sequence include a base sequence designated in official analytical methods or officially announced methods (for example, see SEQ ID NOS. 2 and 3).

The nucleic acid may be a nucleic acid derived from the cells to be used, or a nucleic acid introduced by transgenesis. When a nucleic acid introduced by transgenesis and a plasmid are used as the nucleic acid, it is preferable to confirm that one copy of the nucleic acid is introduced per cell. The method for confirming that one copy of the nucleic acid is introduced is not particularly limited and may be appropriately selected depending on the intended purpose. Examples of the method include a sequencer, a PCR method, and a Southern blotting method.

One kind or two or more kinds of nucleic acids having target base sequences may be introduced by transgenesis. Also in the case of introducing only one kind of a nucleic acid by transgenesis, base sequences of the same kind may be introduced in tandem depending on the intended purpose.

The method for transgenesis is not particularly limited and may be appropriately selected depending on the intended purpose so long as the method can introduce an intended copy number of target base sequences at an intended position. Examples of the method include homologous recombination, CRISPR/Cas9, CRISPR/Cpf1, TALEN, Zinc finger nuclease, Flip-in, and Jump-in. In the case of yeast fungi, homologous recombination is preferable among these methods in terms of a high efficiency and ease of controlling.

—Carrier—

It is preferable to handle the amplifiable reagent in a state of being carried on a carrier. When the amplifiable reagent is a nucleic acid, a preferable form is the nucleic acid being carried (or more preferably encapsulated) by the carrier having a particle shape (carrier particles).

The carrier is not particularly limited and may be appropriately selected depending on the intended purpose. Examples of the carrier include a cell, a resin, liposome, and microcapsule.

—Cells—

A cell means a structural, functional unit that includes an amplifiable reagent (for example, a nucleic acid) and forms an organism.

The cells are not particularly limited and may be appropriately selected depending on the intended purpose. All kinds of cells can be used regardless of whether the cells are eukaryotic cells, prokaryotic cells, multicellular organism cells, and unicellular organism cells. One of these kinds of cells may be used alone or two or more of these kinds of cells may be used in combination.

The eukaryotic cells are not particularly limited and may be appropriately selected depending on the intended purpose. Examples of the eukaryotic cells include animal cells, insect cells, plant cells, fungi, algae, and protozoans. One of these kinds of eukaryotic cells may be used alone or two or more of these kinds of eukaryotic cells may be used in combination. Among these eukaryotic cells, animal cells and fungi are preferable.

Adherent cells may be primary cells directly taken from tissues or organs, or may be cells obtained by passaging primary cells directly taken from tissues or organs a few times. Adherent cells may be appropriately selected depending on the intended purpose. Examples of adherent cells include differentiated cells and undifferentiated cells.

Differentiated cells are not particularly limited and may be appropriately selected depending on the intended purpose. Examples of differentiated cells include: hepatocytes, which are parenchymal cells of a liver; stellate cells; Kupffer cells; endothelial cells such as vascular endothelial cells, sinusoidal endothelial cells, and corneal endothelial cells; fibroblasts; osteoblasts; osteoclasts; periodontal ligament-derived cells; epidermal cells such as epidermal keratinocytes; epithelial cells such as tracheal epithelial cells, intestinal epithelial cells, cervical epithelial cells, and corneal epithelial cells; mammary glandular cells; pericytes; muscle cells such as smooth muscle cells and myocardial cells; renal cells; pancreatic islet cells; nerve cells such as peripheral nerve cells and optic nerve cells; chondrocytes; and bone cells.

Undifferentiated cells are not particularly limited and may be appropriately selected depending on the intended purpose. Examples of undifferentiated cells include: pluripotent stem cells such as embryotic stem cells, which are undifferentiated cells, and mesenchymal stem cells having pluripotency; unipotent stem cells such as vascular endothelial progenitor cells having unipotency; and iPS cells.

Fungi are not particularly limited and may be appropriately selected depending on the intended purpose. Examples of fungi include molds and yeast fungi. One of these kinds of fungi may be used alone or two or more of these kinds of fungi may be used in combination. Among these kinds of fungi, yeast fungi are preferable because the cell cycles are adjustable and monoploids can be used.

The cell cycle means a cell proliferation process in which cells undergo cell division and cells (daughter cells) generated by the cell division become cells (mother cells) that undergo another cell division to generate new daughter cells.

Yeast fungi are not particularly limited and may be appropriately selected depending on the intended purpose. For example, yeast fungi that are synchronously cultured to synchronize at a G0/G1 phase, and fixed at a G1 phase are preferable.

Further, for example, as yeast fungi, Bar1-deficient yeasts with enhanced sensitivity to a pheromone (sex hormone) that controls the cell cycle at a G1 phase are preferable. When yeast fungi are Bar1-deficient yeasts, the abundance ratio of yeast fungi with uncontrolled cell cycles can be reduced. This makes it possible to, for example, prevent a specific nucleic acid from increasing in number in the cells contained in a well.

The prokaryotic cells are not particularly limited and may be appropriately selected depending on the intended purpose. Examples of the prokaryotic cells include eubacteria and archaea. One of these kinds of prokaryotic cells may be used alone or two or more of these kinds of prokaryotic cells may be used in combination.

As the cells, dead cells are preferable. With dead cells, it is possible to prevent occurrence of cell division after fractionation.

As the cells, cells that can emit light upon reception of light are preferable. With cells that can emit light upon reception of light, it is possible to land the cells into wells while having a highly accurate control on the number of cells.

Reception of light means receiving of light.

An optical sensor means a passive sensor configured to collect, with a lens, any light in the range from visible light rays visible by human eyes to near infrared rays, short-wavelength infrared rays, and thermal infrared rays that have longer wavelengths than the visible light rays, to obtain, for example, shapes of target cells in the form of image data.

—Cells that can Emit Light Upon Reception of Light—

The cells that can emit light upon reception of light are not particularly limited and may be appropriately selected depending on the intended purpose so long as the cells can emit light upon reception of light. Examples of the cells include cells stained with a fluorescent dye, cells expressing a fluorescent protein, and cells labeled with a fluorescent-labeled antibody.

A cellular site stained with a fluorescent dye, expressing a fluorescent protein, or labeled with a fluorescent-labeled antibody is not particularly limited. Examples of the cellular site include a whole cell, a cell nucleus, and a cellular membrane.

—Fluorescent Dye—

Examples of the fluorescent dye include fluoresceins, azo dyes, rhodamines, coumarins, pyrenes, cyanines. One of these fluorescent dyes may be used alone or two or more of these fluorescent dyes may be used in combination. Among these fluorescent dyes, fluoresceins, azo dyes, rhodamines, and cyanines are preferable, and eosin, Evans blue, trypan blue, rhodamine 6G, rhodamine B, rhodamine 123, and Cy3 are more preferable.

As the fluorescent dye, a commercially available product may be used. Examples of the commercially available product include product name: EOSIN Y (available from Wako Pure Chemical Industries, Ltd.), product name: EVANS BLUE (available from Wako Pure Chemical Industries, Ltd.), product name: TRYPAN BLUE (available from Wako Pure Chemical Industries, Ltd.), product name: RHODAMINE 6G (available from Wako Pure Chemical Industries, Ltd.), product name: RHODAMINE B (available from Wako Pure Chemical Industries, Ltd.), and product name: RHODAMINE 123 (available from Wako Pure Chemical Industries, Ltd.).

—Fluorescent Protein—

Examples of the fluorescent protein include Sirius, EBFP, ECFP, mTurquoise, TagCFP, AmCyan, mTFP1, MidoriishiCyan, CFP, TurboGFP, AcGFP, TagGFP, Azami-Green, ZsGreen, EmGFP, EGFP, GFP2, HyPer, TagYFP, EYFP, Venus, YFP, PhiYFP, PhiYFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, mCherry, mPlum, PS-CFP, Dendra2, Kaede, EosFP, and KikumeGR. One of these fluorescent proteins may be used alone or two or more of these fluorescent proteins may be used in combination.

—Fluorescent-Labeled Antibody—

The fluorescent-labeled antibody is not particularly limited and may be appropriately selected depending on the intended purpose so long as the fluorescent-labeled antibody is fluorescent-labeled. Examples of the fluorescent-labeled antibody include CD4-FITC and CD8-PE. One of these fluorescent-labeled antibodies may be used alone or two or more of these fluorescent-labeled antibodies may be used in combination.

The volume average particle diameter of the cells is preferably 30 micrometers or less, more preferably 10 micrometers or less, and particularly preferably 7 micrometers or less in a free state. When the volume average particle diameter of the cells is 30 micrometers or less, the cells can be suitably used in an inkjet method or a liquid droplet discharging unit such as a cell sorter.

The volume average particle diameter of the cells can be measured by, for example, a measuring method described below.

Ten microliters is extracted from a produced stained yeast dispersion liquid and poured onto a plastic slide formed of PMMA. Then, with an automated cell counter (product name: COUNTESS AUTOMATED CELL COUNTER, available from Invitrogen), the volume average particle diameter of the cells can be measured. The cell number can be obtained by a similar measuring method.

The concentration of the cells in a cell suspension is not particularly limited, may be appropriately selected depending on the intended purpose, and is preferably 5×10⁴ cells/mL or higher but 5×10⁸ cells/mL or lower and more preferably 5×10⁴ cells/mL or higher but 5×10⁷ cells/mL or lower. When the cell number is 5×10⁴ cells/mL or higher but 5×10⁸ cells/mL or lower, it can be ensured that cells be contained in a discharged liquid droplet without fail. The cell number can be measured with an automated cell counter (product name: COUNTESS AUTOMATED CELL COUNTER, available from Invitrogen) in the same manner as measuring the volume average particle diameter.

The cell number of cells including a nucleic acid is not particularly limited and may be appropriately selected depending on the intended purpose so long as the cell number is a plural number.

—Resin—

The material, the shape, the size, and the structure of the resin are not particularly limited and may be appropriately selected depending on the intended purpose so long as the resin can carry the amplifiable reagent (for example, a nucleic acid).

—Liposome—

A liposome is a lipid vesicle formed of a lipid bilayer containing lipid molecules. Specifically, the liposome means a lipid-containing closed vesicle including a space separated from the external environment by a lipid bilayer produced based on the polarities of a hydrophobic group and a hydrophilic group of lipid molecules.

The liposome is a closed vesicle formed of a lipid bilayer using a lipid, and contains an aqueous phase (internal aqueous phase) in the space in the closed vesicle. The internal aqueous phase contains, for example, water. The liposome may be single-lamellar (single-layer lamellar or unilamellar with a single bilayer) or multilayer lamellar (multilamellar, with an onion-like structure including multiple bilayers, with the individual layers separated by watery layers).

As the liposome, a liposome that can encapsulate an amplifiable reagent (for example, a nucleic acid) is preferable. The form of encapsulation is not particularly limited. “Encapsulation” means a form of a nucleic acid being contained in the internal aqueous phase and the layer of the liposome. Examples of the form include a form of encapsulating a nucleic acid in the closed space formed of the layer, a form of encapsulating a nucleic acid in the layer per se, and a combination of these forms.

The size (average particle diameter) of the liposome is not particularly limited so long as the liposome can encapsulate an amplifiable reagent (for example, a nucleic acid). It is preferable that the liposome have a spherical form or a form close to the spherical form.

The component (layer component) constituting the lipid bilayer of the liposome is selected from lipids. As the lipid, an arbitrary lipid that can dissolve in a mixture solvent of a water-soluble organic solvent and an ester-based organic solvent can be used. Specific examples of the lipid include phospholipids, lipids other than phospholipids, cholesterols, and derivatives of these lipids. These components may be formed of a single kind of a component or a plurality of kinds of components.

—Microcapsule—

A microcapsule means a minute particle having a wall material and a hollow structure, and can encapsulate an amplifiable reagent (for example, a nucleic acid) in the hollow structure.

The microcapsule is not particularly limited, and, for example, the wall material and the size of the microcapsule may be appropriately selected depending on the intended purpose.

Examples of the wall material of the microcapsule include polyurethane resins, polyurea, polyurea-polyurethane resins, urea-formaldehyde resins, melamine-formaldehyde resins, polyamide, polyester, polysulfone amide, polycarbonate, poly-sulfinate, epoxyr, acrylic acid ester, methacrylic acid ester, vinyl acetate, and gelatin. One of these wall materials may be used alone or two or more of these wall materials may be used in combination.

The size of the microcapsule is not particularly limited and may be appropriately selected depending on the intended purpose so long as the microcapsule can encapsulate an amplifiable reagent (for example, a nucleic acid).

The method for producing the microcapsule is not particularly limited and may be appropriately selected depending on the intended purpose. Examples of the method include an in-situ method, an interfacial polymerization method, and a coacervation method.

The detection determining method of the present disclosure and the detection determining device of the present disclosure are suitably used particularly for genetic testing in which the testing target is a virus, a bacterium, or animal species determination of edible meat.

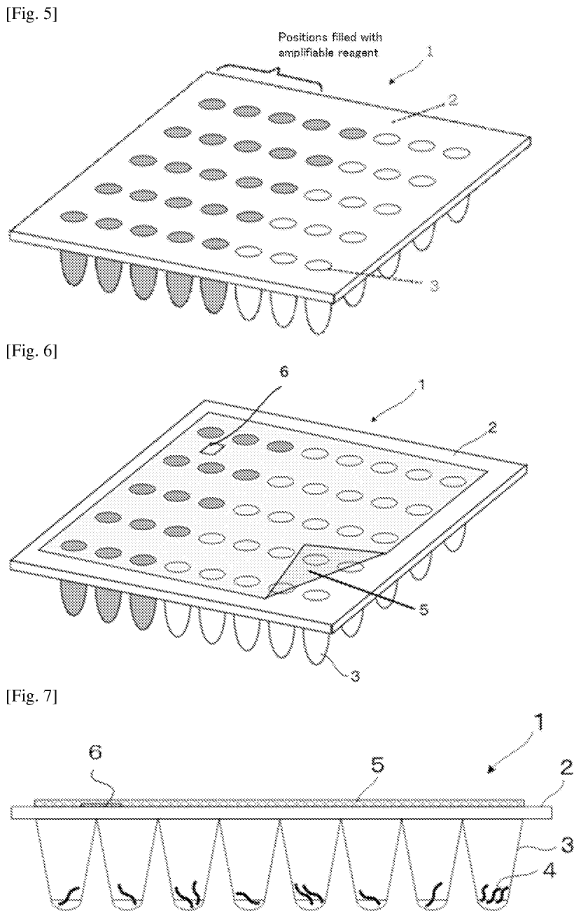

FIG. 5 is a perspective view illustrating an example of the device of the present disclosure. FIG. 6 is a perspective view illustrating another example of the device of the present disclosure. FIG. 7 is a side view of the device of FIG. 6.

In the device 1, a plurality of wells 3 are provided in a base material 2, and a nucleic acid 4 serving as the amplifiable reagent is filled in specific copy numbers in the wells 3 (internal spatial regions enclosed by the well wall surfaces constituting the wells). Information on the absolute numbers of amplifiable reagents and the uncertainty of the absolute numbers of amplifiable reagents is associated with this device 1. FIG. 6 and FIG. 7 illustrate an example in which the openings of the wells 3 of the device 1 are covered with a sealing member 5.

For example, as illustrated in FIG. 6 and FIG. 7, an IC chip or a barcode (identifier unit 6) storing the information on the number of the reagent filled in each well 3 and the uncertainty (or certainty) of the number, or information associated with these kinds of information is placed at a position that is between the sealing member 5 and the base material 2 and does not overlap the openings of the wells. This is suitable for preventing, for example, unintentional alteration of the identifier unit.

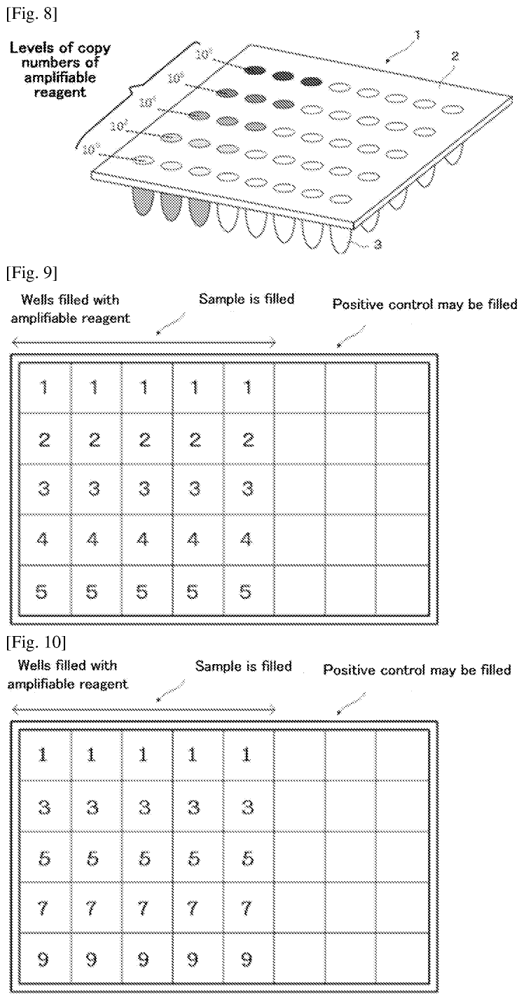

With the identifier unit, the device can be distinguished from a common well plate that does not have an identifier unit. Therefore, confusion or mistake can be prevented. FIG. 8 is a perspective view illustrating another example of the device of the present disclosure. The device of FIG. 8 is provided with five levels of 1, 2, 3, 4, and 5 as the levels of the specific copy number of the amplifiable reagent.

FIG. 9 is a diagram illustrating an example of the positions of the wells to be filled with the amplifiable reagent in the device of the present disclosure. The numerals in the wells in FIG. 9 indicate the specific copy numbers in which the amplifiable reagent is contained. The wells with no numerals in FIG. 9 may be filled with, for example, a positive control.

FIG. 10 is a diagram illustrating another example of the positions of the wells to be filled with the amplifiable reagent in the device of the present disclosure. The numerals in the wells in FIG. 10 indicate the specific copy numbers in which the amplifiable reagent is contained. The wells with no numerals in FIG. 10 may be filled with, for example, a positive control.

The state of the amplifiable reagent, a primer, and an amplifying reagent in the well is not particularly limited and may be appropriately selected depending on the intended purpose. For example, the state of the amplifiable reagent, a primer, and an amplifying reagent may be a state of either a solution or a solid. In terms of convenience of use, the state of the amplifiable reagent, a primer, and an amplifying reagent is particularly preferably a state of a solution. In a state of a solution, a user can use the amplifiable reagent, a primer, and an amplifying reagent for a test immediately. In terms of transportation, the state of the amplifiable reagent, a primer, and an amplifying reagent is particularly preferably a state of a solid and more preferably a dry state. In a solid dry state, a reaction speed at which the amplifiable reagent is decomposed by, for example, a breakdown enzyme, can be reduced, and storage stability of the amplifiable reagent, a primer, and an amplifying reagent can be improved.

It is preferable that the amplifiable reagent, a primer, and an amplifying reagent be filled in appropriate amounts in the device in the solid dry state, in order to make it possible to use the amplifiable reagent, a primer, and an amplifying reagent in the form of a reaction solution immediately by dissolving the amplifiable reagent, a primer, and an amplifying reagent in a buffer or water immediately before use of the device. The drying method is not particularly limited and may be appropriately selected depending on the intended purpose. Examples of the drying method include freeze drying, heating drying, hot-air drying, vacuum drying, steam drying, suction drying, infrared drying, barrel drying, and spin drying.

<Device Producing Method>

A device producing method using cells including a specific nucleic acid as an amplifiable reagent will be described below.

The device producing method of the present disclosure includes a cell suspension producing step of producing a cell suspension containing a plurality of cells including a specific nucleic acid and a solvent, a liquid droplet landing step of discharging the cell suspension in the form of liquid droplets to sequentially land the liquid droplets in wells of a plate, a cell number counting step of counting the number of cells contained in the liquid droplets with a sensor after the liquid droplets are discharged and before the liquid droplets land in the wells, and a nucleic acid extracting step of extracting nucleic acids from cells in the wells, preferably includes a step of calculating the degrees of certainty of estimated numbers of nucleic acids in the cell suspension producing step, the liquid droplet landing step, and the cell number counting step, an outputting step, and a recording step, and further includes other steps as needed.

<<Cell Suspension Producing Method>>

The cell suspension producing step is a step of producing a cell suspension containing a plurality of cells including a specific nucleic acid and a solvent.

The solvent means a liquid used for dispersing cells.

Suspension in the cell suspension means a state of cells being present dispersedly in the solvent.

Producing means a producing operation.

—Cell Suspension—

The cell suspension contains a plurality of cells including a specific nucleic acid and a solvent, preferably contains an additive, and further contains other components as needed.

The plurality of cells including a specific nucleic acid are as described above.

—Solvent—

The solvent is not particularly limited and may be appropriately selected depending on the intended purpose. Examples of the solvent include water, a culture fluid, a separation liquid, a diluent, a buffer, an organic matter dissolving liquid, an organic solvent, a polymeric gel solution, a colloid dispersion liquid, an electrolytic aqueous solution, an inorganic salt aqueous solution, a metal aqueous solution, and mixture liquids of these liquids. One of these solvents may be used alone or two or more of these solvents may be used in combination. Among these solvents, water and a buffer are preferable, and water, a phosphate buffered saline (PBS), and a Tris-EDTA buffer (TE) are more preferable.

—Additive—

An additive is not particularly limited and may be appropriately selected depending on the intended purpose. Examples of the additive include a surfactant, a nucleic acid, and a resin. One of these additives may be used alone or two or more of these additives may be used in combination.

The surfactant can prevent mutual aggregation of cells and improve continuous discharging stability.

The surfactant is not particularly limited and may be appropriately selected depending on the intended purpose. Examples of the surfactant include ionic surfactants and nonionic surfactants. One of these surfactants may be used alone or two or more of these surfactants may be used in combination. Among these surfactants, nonionic surfactants are preferable because proteins are neither modified nor deactivated by nonionic surfactants, although depending on the addition amount of the nonionic surfactants.

Examples of the ionic surfactants include fatty acid sodium, fatty acid potassium, alpha-sulfo fatty acid ester sodium, sodium straight-chain alkyl benzene sulfonate, alkyl sulfuric acid ester sodium, alkyl ether sulfuric acid ester sodium, and sodium alpha-olefin sulfonate. One of these ionic surfactants may be used alone or two or more of these ionic surfactants may be used in combination. Among these ionic surfactants, fatty acid sodium is preferable and sodium dodecyl sulfonate (SDS) is more preferable.

Examples of the nonionic surfactants include alkyl glycoside, alkyl polyoxyethylene ether (e.g., BRIJ series), octyl phenol ethoxylate (e.g., TRITON X series, IGEPAL CA series, NONIDET P series, and NIKKOL OP series), polysorbates (e.g., TWEEN series such as TWEEN 20), sorbitan fatty acid esters, polyoxyethylene fatty acid esters, alkyl maltoside, sucrose fatty acid esters, glycoside fatty acid esters, glycerin fatty acid esters, propylene glycol fatty acid esters, and fatty acid monoglyceride. One of these nonionic surfactants may be used alone or two or more of these nonionic surfactants may be used in combination. Among these nonionic surfactants, polysorbates are preferable.

The content of the surfactant is not particularly limited, may be appropriately selected depending on the intended purpose, and is preferably 0.001% by mass or greater but 30% by mass or less relative to the total amount of the cell suspension. When the content of the surfactant is 0.001% by mass or greater, an effect of adding the surfactant can be obtained. When the content of the surfactant is 30% by mass or less, aggregation of cells can be suppressed, making it possible to strictly control the number of nucleic acid molecules in the cell suspension.

The nucleic acid is not particularly limited and may be appropriately selected depending on the intended purpose so long as the nucleic acid does not affect detection of the detection target nucleic acid. Examples of the nucleic acid include ColE1 DNA. With such a nucleic acid, it is possible to prevent the nucleic acid having a target base sequence from adhering to the wall surface of a well.

The resin is not particularly limited and may be appropriately selected depending on the intended purpose. Examples of the resin include polyethyleneimide.

—Other Materials—

Other materials are not particularly limited and may be appropriately selected depending on the intended purpose. Examples of the other materials include a crosslinking agent, a pH adjustor, an antiseptic, an antioxidant, an osmotic pressure regulator, a humectant, and a dispersant.

<Method for Dispersing Cells>

The method for dispersing the cells is not particularly limited and may be appropriately selected depending on the intended purpose. Examples of the method include a medium method such as a bead mill, an ultrasonic method such as an ultrasonic homogenizer, and a method using a pressure difference such as a French press. One of these methods may be used alone or two or more of these methods may be used in combination. Among these methods, the ultrasonic method is more preferable because the ultrasonic method has low damage on the cells. With the medium method, a high crushing force may destroy cellular membranes or cell walls, and the medium may mix as contamination.

<Method for Screening Cells>

The method for screening the cells is not particularly limited and may be appropriately selected depending on the intended purpose. Examples of the method include screening by wet classification, a cell sorter, and a filter. One of these methods may be used alone or two or more of these methods may be used in combination. Among these methods, screening by a cell sorter and a filter is preferable because the method has low damage on the cells.

It is preferable to estimate the number of nucleic acids having a target base sequence from the cell number contained in the cell suspension, by measuring the cell cycles of the cells.

Measuring the cell cycles means quantifying the cell number due to cell division.

Estimating the number of nucleic acids means obtaining the copy number of nucleic acids based on the cell number.

What is to be counted needs not be the cell number, but may be the number of target base sequences. Typically, it is safe to consider that the number of target base sequences is equal to the cell number, because the cells to be selected as the cells to be counted are cells each including one target base sequence (=one target base sequence per cell), or because one target base sequence is introduced per cell by gene recombination. However, nucleic acid replication occurs in cells in order for the cells to undergo cell division at specific cycles. Cell cycles are different depending on the kinds of cells. By extracting a certain amount of the solution from the cell suspension and measuring the cycles of a plurality of cells, it is possible to calculate an expected value of the number of target base sequences included in one cell and the degree of certainty of the estimated value. This can be realized by, for example, observing nuclear stained cells with a flow cytometer.

Degree of certainty means a probability of occurrence of one specific event, predicted beforehand, when there are possibilities of occurrence of some events.

Calculation means deriving a needed value by a calculating operation.

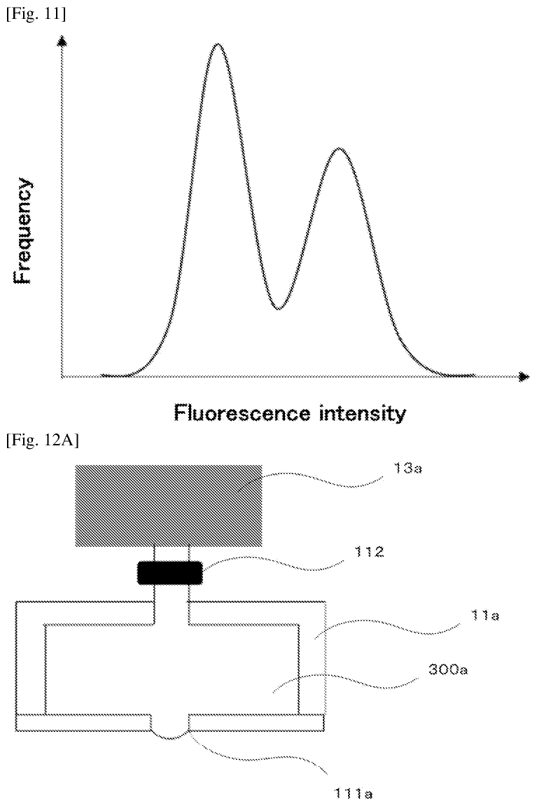

FIG. 11 is a graph plotting an example of a relationship between the frequency and the fluorescence intensity of cells in which target base sequence replication has occurred. As plotted in FIG. 11, based on presence or absence of target base sequence replication, two peaks appear on the histogram. Hence, the percentage of presence of cells in which target base sequence replication has occurred can be calculated. Based on this calculation result, the average target base sequence number included in one cell can be calculated. The estimated number of target base sequences can be calculated by multiplying the counted cell number by the obtained average target base sequence number.

It is preferable to perform an operation of controlling the cell cycles before producing the cell suspension. By preparing the cells uniformly to a state before replication occurs or a state after replication has occurred, it is possible to calculate the number of target base sequences based on the cell number more accurately.

It is preferable to calculate the degree of certainty (probability) for the estimated specific copy number. By calculating the degree of certainty (probability), it is possible to express and output the degree of certainty as a variance or a standard deviation based on these values. When adding up influences of a plurality of factors, it is possible use a square root of the sum of the squares of the standard deviation commonly used. For example, a correct answer percentage for the number of cells discharged, the number of DNA in a cell, and a landing ratio at which discharged cells land in wells can be used as the factors. A highly influential factor may be selected for calculation.

<<Liquid Droplet Landing Step>>

The liquid droplet landing step is a step of discharging the cell suspension in the form of liquid droplets to sequentially land the liquid droplets in wells of a plate. A liquid droplet means a gathering of a liquid formed by a surface tension. Discharging means making the cell suspension fly in the form of liquid droplets. “Sequentially” means “in order”.

Landing means making liquid droplets reach the wells.

As a discharging unit, a unit configured to discharge the cell suspension in the form of liquid droplets (hereinafter may also be referred to as “discharging head”) can be suitably used.

Examples of the method for discharging the cell suspension in the form of liquid droplets include an on-demand method and a continuous method that are based on the inkjet method. Of these methods, in the case of the continuous method, there is a tendency that the dead volume of the cell suspension used is high, because of, for example, empty discharging until the discharging state becomes stable, adjustment of the amount of liquid droplets, and continued formation of liquid droplets even during transfer between the wells. In the present disclosure, in terms of cell number adjustment, it is preferable to suppress influence due to the dead volume. Hence, of the two methods, the on-demand method is more preferable.

Examples of the on-demand method include a plurality of known methods such as a pressure applying method of applying a pressure to a liquid to discharge the liquid, a thermal method of discharging a liquid by film boiling due to heating, and an electrostatic method of drawing liquid droplets by electrostatic attraction to form liquid droplets. Among these methods, the pressure applying method is preferable for the reason described below.

In the electrostatic method, there is a need for disposing an electrode in a manner to face a discharging unit that is configured to retain the cell suspension and form liquid droplets. In the device producing method, a plate for receiving liquid droplets is disposed at the facing position. Hence, it is preferable not to provide an electrode, in order to increase the degree of latitude in the plate configuration.

In the thermal method, there are a risk of local heating concentration that may affect the cells, which are a biomaterial, and a risk of kogation to the heater portion. Influences by heat depend on the components contained or the purpose for which the plate is used. Therefore, there is no need for flatly rejecting the thermal method. However, the pressure applying method is preferable because the pressure applying method has a lower risk of kogation to the heater portion than the thermal method.

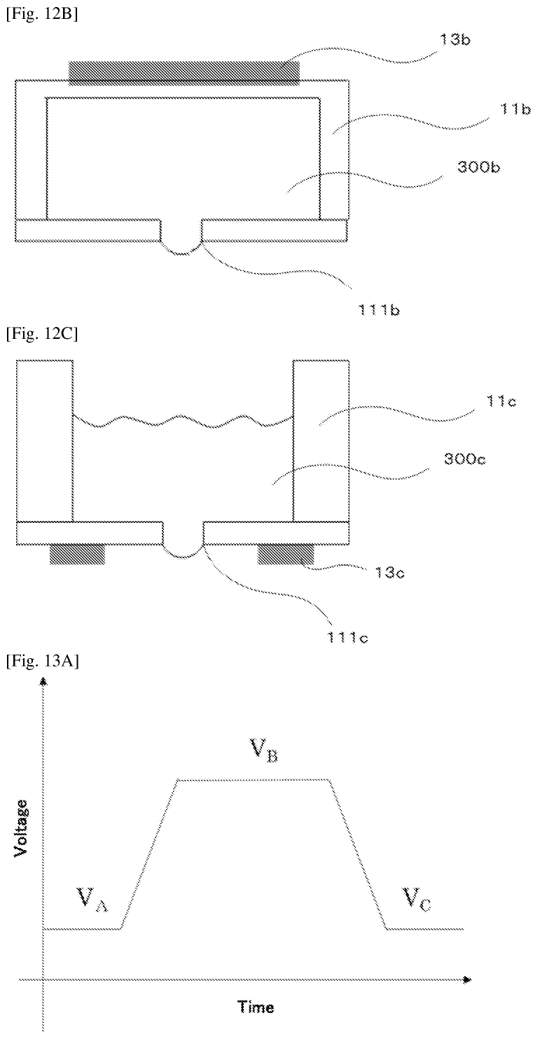

Examples of the pressure applying method include a method of applying a pressure to a liquid using a piezo element, and a method of applying a pressure using a valve such as an electromagnetic valve. The configuration example of a liquid droplet generating device usable for discharging liquid droplets of the cell suspension is illustrated in FIG. 12A to FIG. 12C.

FIG. 12A is an exemplary diagram illustrating an example of an electromagnetic valve-type discharging head. The electromagnetic valve-type discharging head includes an electric motor 13a, an electromagnetic valve 112, a liquid chamber 11a, a cell suspension 300a, and a nozzle 111a.

As the electromagnetic valve-type discharging head, for example, a dispenser available from Tech Elan LLC can be suitably used.

FIG. 12B is an exemplary diagram illustrating an example of a piezo-type discharging head. The piezo-type discharging head includes a piezoelectric element 13b, a liquid chamber 11b, a cell suspension 300b, and a nozzle 111b.

As the piezo-type discharging head, for example, a single cell printer available from Cytena GmbH can be suitably used.

Any of these discharging heads may be used. However, the pressure applying method by the electromagnetic valve is not capable of forming liquid droplets at a high speed repeatedly. Therefore, it is preferable to use the piezo method in order to increase the throughput of producing a plate. A piezo-type discharging head using a common piezoelectric element 13b may cause unevenness in the cell concentration due to settlement, or may have nozzle clogging.

Therefore, a more preferable configuration is the configuration illustrated in FIG. 12C. FIG. 12C is an exemplary diagram of a modified example of a piezo-type discharging head using the piezoelectric element illustrated in FIG. 12B. The discharging head of FIG. 12C includes a piezoelectric element 13c, a liquid chamber 11c, a cell suspension 300c, and a nozzle 111c.

In the discharging head of FIG. 12C, when a voltage is applied to the piezoelectric element 13c from an unillustrated control device, a compressive stress is applied in the horizontal direction of the drawing sheet. This can deform the membrane in the upward-downward direction of the drawing sheet.

Examples of any other method than the on-demand method include a continuous method for continuously forming liquid droplets. When pushing out liquid droplets from a nozzle by pressurization, the continuous method applies regular fluctuations using a piezoelectric element or a heater, to make it possible to continuously form minute liquid droplets. Further, the continuous method can select whether to land a flying liquid droplet into a well or to recover the liquid droplet in a recovery unit, by controlling the discharging direction of the liquid droplet with voltage application. Such a method is employed in a cell sorter or a flow cytometer. For example, a device named: CELL SORTER SH800Z available from Sony Corporation can be used.

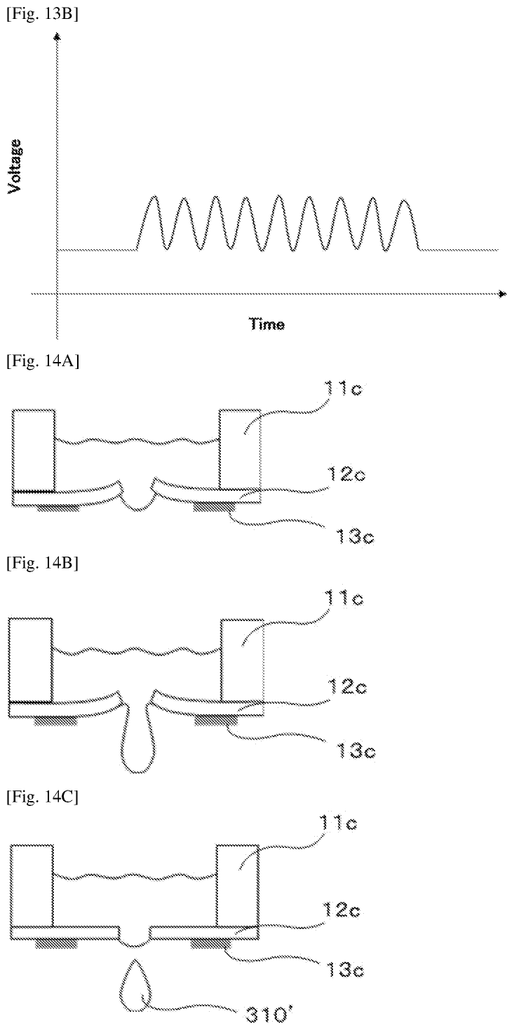

FIG. 13A is an exemplary graph plotting an example of a voltage applied to a piezoelectric element. FIG. 13B is an exemplary graph plotting another example of a voltage applied to a piezoelectric element. FIG. 13A plots a drive voltage for forming liquid droplets. Depending on the high or low level of the voltage (V_{A}, V_{B}, and V_{C}), it is possible to form liquid droplets. FIG. 13B plots a voltage for stirring the cell suspension without discharging liquid droplets.

During a period in which liquid droplets are not discharged, inputting a plurality of pulses that are not high enough to discharge liquid droplets enables the cell suspension in the liquid chamber to be stirred, making it possible to suppress occurrence of a concentration distribution due to settlement of the cells.

The liquid droplet forming operation of the discharging head that can be used in the present disclosure will be described below.

The discharging head can discharge liquid droplets with application of a pulsed voltage to the upper and lower electrodes formed on the piezoelectric element. FIG. 14A to FIG. 14C are exemplary diagrams illustrating liquid droplet states at the respective timings.

In FIG. 14A, first, upon application of a voltage to the piezoelectric element 13c, a membrane 12c abruptly deforms to cause a high pressure between the cell suspension retained in the liquid chamber 11c and the membrane 12c. This pressure pushes out a liquid droplet outward through the nozzle portion.

Next, as illustrated in FIG. 14B, for a period of time until when the pressure relaxes upward, the liquid is continuously pushed out through the nozzle portion, to grow the liquid droplet.

Finally, as illustrated in FIG. 14C, when the membrane 12c returns to the original state, the liquid pressure about the interface between the cell suspension and the membrane 12c lowers, to form a liquid droplet 310′.

In the device producing method, a plate in which wells are formed is secured on a movable stage, and by combination of driving of the stage with formation of liquid droplets from the discharging head, liquid droplets are sequentially landed in the concaves. A method of moving the plate along with moving the stage is described here. However, naturally, it is also possible to move the discharging head.

The plate is not particularly limited, and a plate that is commonly used in bio fields and in which wells are formed can be used.

The number of wells in the plate is not particularly limited and may be appropriately selected depending on the intended purpose. The number of wells may be a single number or a plural number.

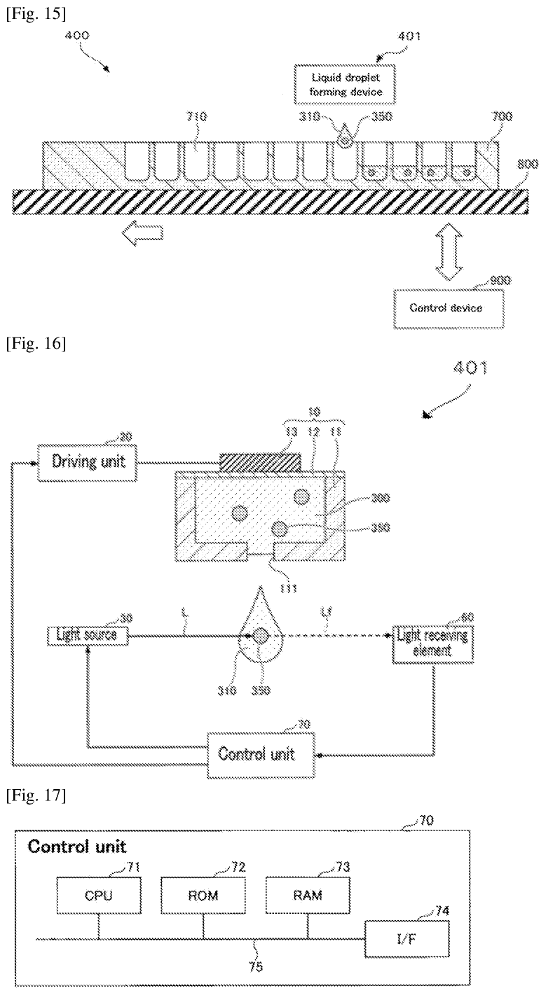

FIG. 15 is a schematic diagram illustrating an example of a dispensing device 400 configured to land liquid droplets sequentially into wells of a plate.

As illustrated in FIG. 15, the dispensing device 400 configured to land liquid droplets includes a liquid droplet forming device 401, a plate 700, a stage 800, and a control device 900.

In the dispensing device 400, the plate 700 is disposed over a movable stage 800. The plate 700 has a plurality of wells 710 (concaves) in which liquid droplets 310 discharged from a discharging head of the liquid droplet forming device 401 land. The control device 900 is configured to move the stage 800 and control the relative positional relationship between the discharging head of the liquid droplet forming device 401 and each well 710. This enables liquid droplets 310 containing fluorescent-stained cells 350 to be discharged sequentially into the wells 710 from the discharging head of the liquid droplet forming device 401.

The control device 900 may be configured to include, for example, a CPU, a ROM, a RAM, and a main memory. In this case, various functions of the control device 900 can be realized by a program recorded in, for example, the ROM being read out into the main memory and executed by the CPU. However, a part or the whole of the control device 900 may be realized only by hardware. Alternatively, the control device 900 may be configured with, for example, physically a plurality of devices.

When landing the cell suspension into the wells, it is preferable to land the liquid droplets to be discharged into the wells, in a manner that a plurality of levels are obtained.

A plurality of levels mean a plurality of references serving as standards.

As the plurality of levels, it is preferable that a plurality of cells including a specific nucleic acid have a predetermined concentration gradient in the wells. With a concentration gradient, the nucleic acid can be favorably used as a reagent for calibration curve. The plurality of levels can be controlled using values counted by a sensor.

As the plate, it is preferable to use, for example, a 1-well microtube, 8-series tubes, a 96-well plate, and a 384-well plate. When the number of wells are a plural number, it is possible to dispense the same number of cells into the wells of these plates, or it is also possible to dispense numbers of cells of different levels into the wells. There may be a well in which no cells are contained. Particularly, for producing a plate used for evaluating a real-time PCR device or digital PCR device configured to quantitatively evaluate an amount of nucleic acids, it is preferable to dispense numbers of nucleic acids of a plurality of levels. For example, it is conceivable to produce a plate into which cells (or nucleic acids) are dispensed at 7 levels, namely about 1 cell, 2 cells, 4 cells, 8 cells, 16 cells, 32 cells, and 64 cells. Using such a plate, it is possible to inspect, for example, quantitativity, linearity, and lower limit of evaluation of a real-time PCR device or digital PCR device.

<<Cell Number Counting Step>>

The cell number counting step is a step of counting the number of cells contained in the liquid droplets with a sensor after the liquid droplets are discharged and before the liquid droplets land in the wells.

A sensor means a device configured to, by utilizing some scientific principles, change mechanical, electromagnetic, thermal, acoustic, or chemical properties of natural phenomena or artificial products or spatial information/temporal information indicated by these properties into signals, which are a different medium easily handleable by humans or machines.

Counting means counting of numbers.

The cell number counting step is not particularly limited and may be appropriately selected depending on the intended purpose, so long as the cell number counting step counts the number of cells contained in the liquid droplets with a sensor after the liquid droplets are discharged and before the liquid droplets land in the wells. The cell number counting step may include an operation for observing cells before discharging and an operation for counting cells after landing.

For counting the number of cells contained in the liquid droplets after the liquid droplets are discharged and before the liquid droplets land in the wells, it is preferable to observe cells in a liquid droplet at a timing at which the liquid droplet is at a position that is immediately above a well opening and at which the liquid droplet is predicted to enter the well in the plate without fail.

Examples of the method for observing cells in a liquid droplet include an optical detection method and an electric or magnetic detection method.

—Optical Detection Method—

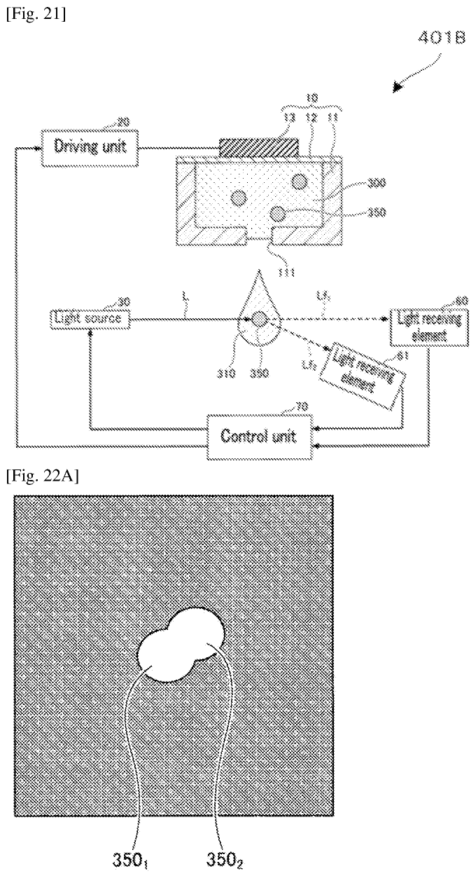

With reference to FIG. 16, FIG. 20, and FIG. 21, an optical detection method will be described below.

FIG. 16 is an exemplary diagram illustrating an example of a liquid droplet forming device 401. FIG. 20 and FIG. 21 are exemplary diagrams illustrating other examples of liquid droplet forming devices 401A and 401B. As illustrated in FIG. 16, the liquid droplet forming device 401 includes a discharging head (liquid droplet discharging unit) 10, a driving unit 20, a light source 30, a light receiving element 60, and a control unit 70.

In FIG. 16, a liquid obtained by dispersing cells in a predetermined solution after fluorescently staining the cells with a specific pigment is used as the cell suspension. Cells are counted by irradiating the liquid droplets formed by the discharging head with light having a specific wavelength and emitted from the light source and detecting fluorescence emitted by the cells with the light receiving element. Here, autofluorescence emitted by molecules originally contained in the cells may be utilized, in addition to the method of staining the cells with a fluorescent pigment. Alternatively, genes for producing fluorescent proteins (for example, GFP (Green Fluorescent Proteins)) may be previously introduced into the cells, in order that the cells may emit fluorescence.

Irradiation of light means application of light.

The discharging head 10 includes a liquid chamber 11, a membrane 12, and a driving element 13 and can discharge a cell suspension 300 suspending fluorescent-stained cells 350 in the form of liquid droplets.

The liquid chamber 11 is a liquid retaining portion configured to retain the cell suspension 300 suspending the fluorescent-stained cells 350. A nozzle 111, which is a through hole, is formed in the lower surface of the liquid chamber 11. The liquid chamber 11 may be formed of, for example, a metal, silicon, or a ceramic. Examples of the fluorescent-stained cells 350 include inorganic particles and organic polymer particles stained with a fluorescent pigment.

The membrane 12 is a film-shaped member secured on the upper end portion of the liquid chamber 11. The planar shape of the membrane 12 may be, for example, a circular shape, but may also be, for example, an elliptic shape or a quadrangular shape.

The driving element 13 is provided on the upper surface of the membrane 12. The shape of the driving element 13 may be designed to match the shape of the membrane 12. For example, when the planar shape of the membrane 12 is a circular shape, it is preferable to provide a circular driving element 13.

The membrane 12 can be vibrated by supplying a driving signal to the driving element 13 from a driving unit 20. The vibration of the membrane 12 can cause a liquid droplet 310 containing the fluorescent-stained cells 350 to be discharged through the nozzle 111.

When a piezoelectric element is used as the driving element 13, for example, the driving element 13 may have a structure obtained by providing the upper surface and the lower surface of the piezoelectric material with electrodes across which a voltage is to be applied. In this case, when the driving unit 20 applies a voltage across the upper and lower electrodes of the piezoelectric element, a compressive stress is applied in the horizontal direction of the drawing sheet, making it possible for the membrane 12 to vibrate in the upward-downward direction of the drawing sheet. As the piezoelectric material, for example, lead zirconate titanate (PZT) may be used. In addition, various piezoelectric materials can be used, such as bismuth iron oxide, metal niobate, barium titanate, or materials obtained by adding metals or different oxides to these materials.

The light source 30 is configured to irradiate a flying liquid droplet 310 with light L. A flying state means a state from when the liquid droplet 310 is discharged from a liquid droplet discharging unit 10 until when the liquid droplet 310 lands on the landing target. A flying liquid droplet 310 has an approximately spherical shape at the position at which the liquid droplet 310 is irradiated with the light L. The beam shape of the light L is an approximately circular shape.

It is preferable that the beam diameter of the light L be from about 10 times through 100 times as great as the diameter of the liquid droplet 310. This is for ensuring that the liquid droplet 310 is irradiated with the light L from the light source 30 without fail even when the position of the liquid droplet 310 fluctuates.

However, it is not preferable if the beam diameter of the light L is much greater than 100 times as great as the diameter of the liquid droplet 310. This is because the energy density of the light with which the liquid droplet 310 is irradiated is reduced, to lower the light volume of fluorescence Lf to be emitted upon the light L serving as excitation light, making it difficult for the light receiving element 60 to detect the fluorescence Lf.

It is preferable that the light L emitted by the light source 30 be pulse light. It is preferable to use, for example, a solid-state laser, a semiconductor laser, and a dye laser. When the light L is pulse light, the pulse width is preferably 10 microseconds or less and more preferably 1 microsecond or less. The energy per unit pulse is preferably roughly 0.1 microjoules or higher and more preferably 1 microjoule or higher, although significantly depending on the optical system such as presence or absence of light condensation.

The light receiving element 60 is configured to receive fluorescence Lf emitted by the fluorescent-stained cell 350 upon absorption of the light L as excitation light, when the fluorescent-stained cell 350 is contained in a flying liquid droplet 310. Because the fluorescence Lf is emitted to all directions from the fluorescent-stained cell 350, the light receiving element 60 can be disposed at an arbitrary position at which the fluorescence Lf is receivable. Here, in order to improve contrast, it is preferable to dispose the light receiving element 60 at a position at which direct incidence of the light L emitted by the light source 30 to the light receiving element 60 does not occur.

The light receiving element 60 is not particularly limited and may be appropriately selected depending on the intended purpose so long as the light receiving element 60 is an element capable of receiving the fluorescence Lf emitted by the fluorescent-stained cell 350. An optical sensor configured to receive fluorescence from a cell in a liquid droplet when the liquid droplet is irradiated with light having a specific wavelength is preferable. Examples of the light receiving element 60 include one-dimensional elements such as a photodiode and a photosensor. When high-sensitivity measurement is needed, it is preferable to use a photomultiplier tube and an Avalanche photodiode. As the light receiving element 60, two-dimensional elements such as a CCD (Charge Coupled Device), a CMOS (Complementary Metal Oxide Semiconductor), and a gate CCD may be used.

The fluorescence Lf emitted by the fluorescent-stained cell 350 is weaker than the light L emitted by the light source 30. Therefore, a filter configured to attenuate the wavelength range of the light L may be installed at a preceding stage (light receiving surface side) of the light receiving element 60. This enables the light receiving element 60 to obtain an extremely highly contrastive image of the fluorescent-stained cell 350. As the filter, for example, a notch filter configured to attenuate a specific wavelength range including the wavelength of the light L may be used.

As described above, it is preferable that the light L emitted by the light source 30 be pulse light. The light L emitted by the light source 30 may be continuously oscillating light. In this case, it is preferable to control the light receiving element 60 to be capable of receiving light at a timing at which a flying liquid droplet 310 is irradiated with the continuously oscillating light, to make the light receiving element 60 receive the fluorescence Lf.

The control unit 70 has a function of controlling the driving unit 20 and the light source 30. The control unit 70 also has a function of obtaining information that is based on the light volume received by the light receiving element 60 and counting the number of fluorescent-stained cells 350 contained in the liquid droplet 310 (the case where the number is zero is also included). With reference to FIG. 17 to FIG. 19, an operation of the liquid droplet forming device 401 including an operation of the control unit 70 will be described below.

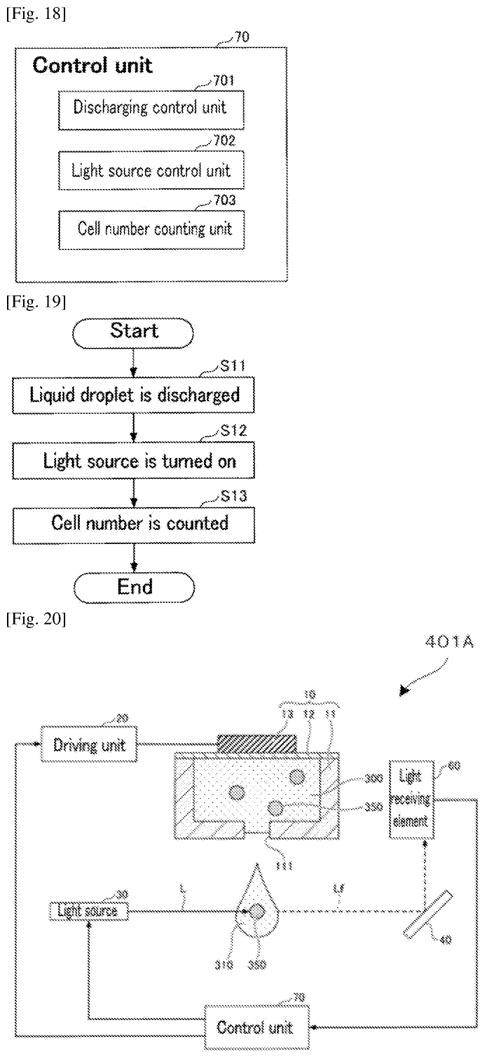

FIG. 17 is a diagram illustrating hardware blocks of the control unit of the liquid droplet forming device of FIG. 16. FIG. 18 is a diagram illustrating functional blocks of the control unit of the liquid droplet forming device of FIG. 16. FIG. 19 is a flowchart illustrating an example of the operation of the liquid droplet forming device.

As illustrated in FIG. 17, the control unit 70 includes a CPU 71, a ROM 72, a RAM 73, an I/F 74, and a bus line 75. The CPU 71, the ROM 72, the RAM 73, and the I/F 74 are coupled to one another via the bus line 75.

The CPU 71 is configured to control various functions of the control unit 70. The ROM 72 serving as a memory unit is configured to store programs to be executed by the CPU 71 for controlling the various functions of the control unit 70 and various information. The RAM 73 serving as a memory unit is configured to be used as, for example, the work area of the CPU 71. The RAM 73 is also configured to be capable of storing predetermined information for a temporary period of time. The I/F 74 is an interface configured to couple the liquid droplet forming device 401 to, for example, another device. The liquid droplet forming device 401 may be coupled to, for example, an external network via the I/F 74.

As illustrated in FIG. 18, the control unit 70 includes a discharging control unit 701, a light source control unit 702, and a cell number counting unit (cell number sensing unit) 703 as functional blocks.

With reference to FIG. 18 and FIG. 19, cell number (particle number) counting by the liquid droplet forming device 401 will be described.

In the step S11, the discharging control unit 701 of the control unit 70 outputs an instruction for discharging to the driving unit 20. Upon reception of the instruction for discharging from the discharging control unit 701, the driving unit 20 supplies a driving signal to the driving element 13 to vibrate the membrane 12. The vibration of the membrane 12 causes a liquid droplet 310 containing a fluorescent-stained cell 350 to be discharged through the nozzle 111.

Next, in the step S12, the light source control unit 702 of the control unit 70 outputs an instruction for lighting to the light source 30 in synchronization with the discharging of the liquid droplet 310 (in synchronization with a driving signal supplied by the driving unit 20 to the liquid droplet discharging unit 10). In accordance with this instruction, the light source 30 is turned on to irradiate the flying liquid droplet 310 with the light L.

Here, the light is emitted by the light source 30, not in synchronization with discharging of the liquid droplet 310 by the liquid droplet discharging unit 10 (supplying of the driving signal to the liquid droplet discharging unit 10 by the driving unit 20), but in synchronization with the timing at which the liquid droplet 310 has come flying to a predetermined position in order for the liquid droplet 310 to be irradiated with the light L. That is, the light source control unit 702 controls the light source 30 to emit light at a predetermined period of time of delay from the discharging of the liquid droplet 310 by the liquid droplet discharging unit 10 (from the driving signal supplied by the driving unit 20 to the liquid droplet discharging unit 10).

For example, the speed v of the liquid droplet 310 to be discharged when the driving signal is supplied to the liquid droplet discharging unit 10 may be measured beforehand. Based on the measured speed v, the time t taken from when the liquid droplet 310 is discharged until when the liquid droplet 310 reaches the predetermined position may be calculated, in order that the timing of light irradiation by the light source 30 may be delayed from the timing at which the driving signal is supplied to the liquid droplet discharging unit 10 by the period of time of t. This enables a good control on light emission, and can ensure that the liquid droplet 310 is irradiated with the light from the light source 30 without fail.

Next, in the step S13, the cell number counting unit 703 of the control unit 70 counts the number of fluorescent-stained cells 350 contained in the liquid droplet 310 (the case where the number is zero is also included) based on information from the light receiving element 60. The information from the light receiving element 60 indicates the luminance (light volume) and the area value of the fluorescent-stained cell 350.

The cell number counting unit 703 can count the number of fluorescent-stained cells 350 by, for example, comparing the light volume received by the light receiving element 60 with a predetermined threshold. In this case, a one-dimensional element may be used or a two-dimensional element may be used as the light receiving element 60.

When a two-dimensional element is used as the light receiving element 60, the cell number counting unit 703 may use a method of performing image processing for calculating the luminance or the area of the fluorescent-stained cell 350 based on a two-dimensional image obtained from the light receiving element 60. In this case, the cell number counting unit 703 can count the number of fluorescent-stained cells 350 by calculating the luminance or the area value of the fluorescent-stained cell 350 by image processing and comparing the calculated luminance or area value with a predetermined threshold.

The fluorescent-stained cell 350 may be a cell or a stained cell. A stained cell means a cell stained with a fluorescent pigment or a cell that can express a fluorescent protein.

The fluorescent pigment for the stained cell is not particularly limited and may be appropriately selected depending on the intended purpose. Examples of the fluorescent pigment include fluoresceins, rhodamines, coumarins, pyrenes, cyanines, and azo pigments. One of these fluorescent pigments may be used alone or two or more of these fluorescent pigments may be used in combination. Among these fluorescent pigments, eosin, Evans blue, trypan blue, rhodamine 6G, rhodamine B, and Rhodamine 123 are more preferable.

Examples of the fluorescent protein include Sirius, EBFP, ECFP, mTurquoise, TagCFP, AmCyan, mTFP1, MidoriishiCyan, CFP, TurboGFP, AcGFP, TagGFP, Azami-Green, ZsGreen, EmGFP, EGFP, GFP2, HyPer, TagYFP, EYFP, Venus, YFP, PhiYFP, PhiYFP-m, TurboYFP, ZsYellow, mBanana, KusabiraOrange, mOrange, TurboRFP, DsRed-Express, DsRed2, TagRFP, DsRed-Monomer, AsRed2, mStrawberry, TurboFP602, mRFP1, JRed, KillerRed, mCherry, mPlum, PS-CFP, Dendra2, Kaede, EosFP, and KikumeGR. One of these fluorescent proteins may be used alone or two or more of these fluorescent proteins may be used in combination.

In this way, in the liquid droplet forming device 401, the driving unit 20 supplies a driving signal to the liquid droplet discharging unit 10 retaining the cell suspension 300 suspending fluorescent-stained cells 350 to cause the liquid droplet discharging unit 10 to discharge a liquid droplet 310 containing the fluorescent-stained cell 350, and the flying liquid droplet 310 is irradiated with the light L from the light source 30. Then, the fluorescent-stained cell 350 contained in the flying liquid droplet 310 emits the fluorescence Lf upon the light L serving as excitation light, and the light receiving element 60 receives the fluorescence Lf. Then, the cell number counting unit 703 counts the number of fluorescent-stained cells 350 contained in the flying liquid droplet 310, based on information from the light receiving element 60.

That is, the liquid droplet forming device 401 is configured for on-the-spot actual observation of the number of fluorescent-stained cells 350 contained in the flying liquid droplet 310. This can realize a better accuracy than hitherto obtained, in counting the number of fluorescent-stained cells 350. Moreover, because the fluorescent-stained cell 350 contained in the flying liquid droplet 310 is irradiated with the light L and emits the fluorescence Lf that is to be received by the light receiving element 60, an image of the fluorescent-stained cell 350 can be obtained with a high contrast, and the frequency of occurrence of erroneous counting of the number of fluorescent-stained cells 350 can be reduced.

FIG. 20 is an exemplary diagram illustrating a modified example of the liquid droplet forming device 401 of FIG. 16. As illustrated in FIG. 20, a liquid droplet forming device 401A is different from the liquid droplet forming device 401 (see FIG. 16) in that a mirror 40 is arranged at the preceding stage of the light receiving element 60. Description about components that are the same as in the embodiment already described may be skipped.

In the liquid droplet forming device 401A, arranging the mirror 40 at the perceiving stage of the light receiving element 60 can improve the degree of latitude in the layout of the light receiving element 60.

For example, in the layout of FIG. 16, when a nozzle 111 and a landing target are brought close to each other, there is a risk of occurrence of interference between the landing target and the optical system (particularly, the light receiving element 60) of the liquid droplet forming device 401. With the layout of FIG. 20, occurrence of interference can be avoided.

That is, by changing the layout of the light receiving element 60 as illustrated in FIG. 20, it is possible to reduce the distance (gap) between the landing target on which a liquid droplet 310 is landed and the nozzle 111 and suppress landing on a wrong position. As a result, the dispensing accuracy can be improved.

FIG. 21 is an exemplary diagram illustrating another modified example of the liquid droplet forming device 401 of FIG. 16. As illustrated in FIG. 21, a liquid droplet forming device 401B is different from the liquid droplet forming device 401 (see FIG. 16) in that a light receiving element 61 configured to receive fluorescence Lf₂ emitted by the fluorescent-stained cell 350 is provided in addition to the light receiving element 60 configured to receive fluorescence Lf₁ emitted by the fluorescent-stained cell 350. Description about components that are the same as in the embodiment already described may be skipped.

The fluorescences Lf₁ and Lf₂ represent parts of fluorescence emitted to all directions from the fluorescent-stained cell 350. The light receiving elements 60 and 61 can be disposed at arbitrary positions at which the fluorescence emitted to different directions by the fluorescent-stained cell 350 is receivable. Three or more light receiving elements may be disposed at positions at which the fluorescence emitted to different directions by the fluorescent-stained cell 350 is receivable. The light receiving elements may have the same specifications or different specifications.

With one light receiving element, when a plurality of fluorescent-stained cells 350 are contained in a flying liquid droplet 310, there is a risk that the cell number counting unit 703 may erroneously count the number of fluorescent-stained cells 350 contained in the liquid droplet 310 (a risk that a counting error may occur) because the fluorescent-stained cells 350 may overlap each other.

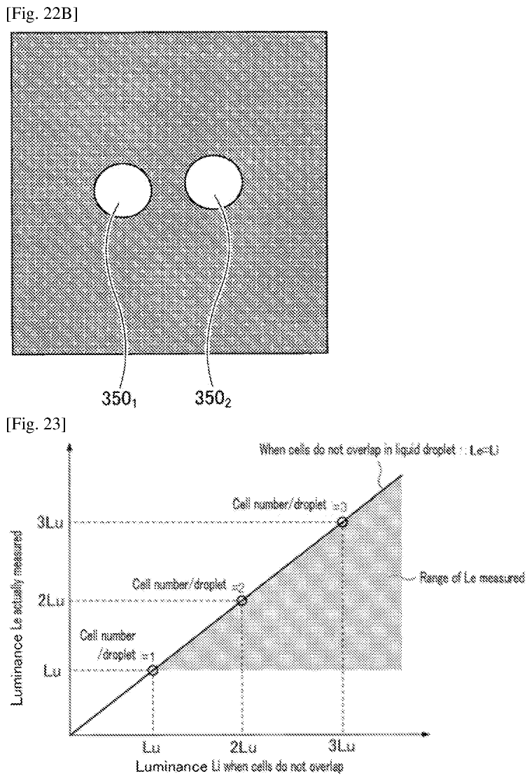

FIG. 22A and FIG. 22B are diagrams illustrating a case where two fluorescent-stained cells are contained in a flying liquid droplet. For example, as illustrated in FIG. 22A, there may be a case where fluorescent-stained cells 350₁ and 350₂ overlap each other, or as illustrated in FIG. 22B, there may be a case where the fluorescent-stained cells 350₁ and 350₂ do not overlap each other. By providing two or more light receiving elements, it is possible to reduce the influence of overlap of the fluorescent-stained cells.

As described above, the cell number counting unit 703 can count the number of fluorescent particles, by calculating the luminance or the area value of fluorescent particles by image processing and comparing the calculated luminance or area value with a predetermined threshold.

When two or more light receiving elements are installed, it is possible to suppress occurrence of a counting error, by adopting the data indicating the maximum value among the luminance values or area values obtained from these light receiving elements. This will be described in more detail with reference to FIG. 23.

FIG. 23 is a graph plotting an example of a relationship between a luminance Li when particles do not overlap each other and a luminance Le actually measured. As plotted in FIG. 23, when particles in the liquid droplet do not overlap each other, Le is equal to Li. For example, in the case where the luminance of one cell is assumed to be Lu, Le is equal to Lu when the number of cells per droplet is 1, and Le is equal to nLu when the number of particles per droplet is n (n: natural number).

However, actually, when n is 2 or greater, because particles may overlap each other, the luminance to be actually measured is Lu≤Le≤nLu (the half-tone dot meshed portion in FIG. 23). Hence, when the number of cells per droplet is n, the threshold may be set to, for example, (nLu−Lu/2)≤threshold<(nLu+Lu/2). When a plurality of light receiving elements are installed, it is possible to suppress occurrence of a counting error, by adopting the maximum value among the data obtained from these light receiving elements. An area value may be used instead of luminance.

When a plurality of light receiving elements are installed, the number of particles may be determined according to an algorithm for estimating the number of cells based on a plurality of shape data to be obtained.

As can be understood, with the plurality of light receiving elements configured to receive fluorescence emitted to different directions by the fluorescent-stained cell 350, the liquid droplet forming device 401B can further reduce the frequency of occurrence of erroneous counting of the number of fluorescent-stained cells 350.

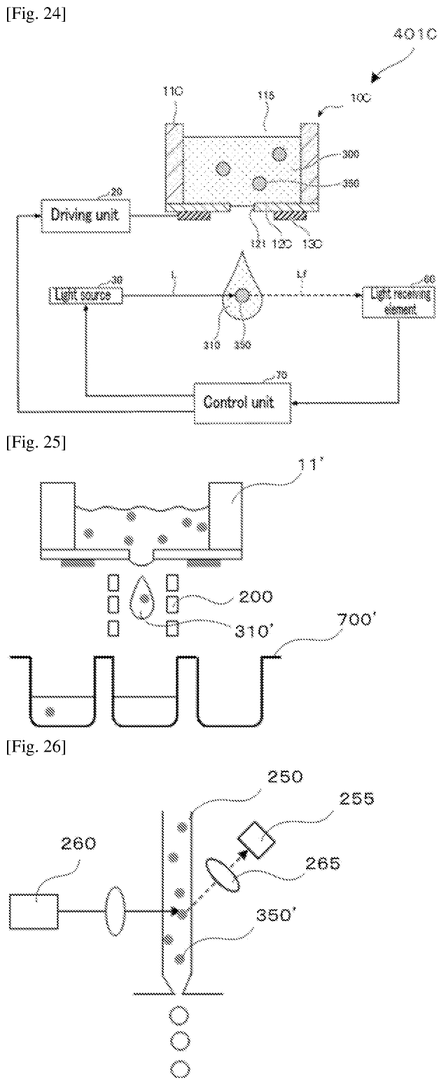

FIG. 24 is an exemplary diagram illustrating another modified example of the liquid droplet forming device 401 of FIG. 16. As illustrated in FIG. 24, a liquid droplet forming device 401C is different from the liquid droplet forming device 401 (see FIG. 16) in that a liquid droplet discharging unit 10C is provided instead of the liquid droplet discharging unit 10. Description about components that are the same as in the embodiment already described may be skipped.

The liquid droplet discharging unit 10C includes a liquid chamber 11C, a membrane 12C, and a driving element 13C. At the top, the liquid chamber 11C has an atmospherically exposed portion 115 configured to expose the interior of the liquid chamber 11C to the atmosphere, and bubbles mixed in the cell suspension 300 can be evacuated through the atmospherically exposed portion 115.

The membrane 12C is a film-shaped member secured at the lower end of the liquid chamber 11C. A nozzle 121, which is a through hole, is formed in approximately the center of the membrane 12C, and the vibration of the membrane 12C causes the cell suspension 300 retained in the liquid chamber 11C to be discharged through the nozzle 121 in the form of a liquid droplet 310. Because the liquid droplet 310 is formed by the inertia of the vibration of the membrane 12C, it is possible to discharge the cell suspension 300 even when the cell suspension 300 has a high surface tension (a high viscosity). The planer shape of the membrane 12C may be, for example, a circular shape, but may also be, for example, an elliptic shape or a quadrangular shape.

The material of the membrane 12C is not particularly limited. However, if the material of the membrane 12C is extremely flexible, the membrane 12C easily undergo vibration and is not easily able to stop vibration immediately when there is no need for discharging. Therefore, a material having a certain degree of hardness is preferable. As the material of the membrane 12C, for example, a metal material, a ceramic material, and a polymeric material having a certain degree of hardness can be used.

Particularly, when a cell is used as the fluorescent-stained cell 350, the material of the membrane is preferably a material having a low adhesiveness with the cell or proteins. Generally, adhesiveness of cells is said to be dependent on the contact angle of the material with respect to water. When the material has a high hydrophilicity or a high hydrophobicity, the material has a low adhesiveness with cells. As the material having a high hydrophilicity, various metal materials and ceramics (metal oxides) can be used. As the material having a high hydrophobicity, for example, fluororesins can be used.

Other examples of such materials include stainless steel, nickel, and aluminum, and silicon dioxide, alumina, and zirconia. In addition, it is conceivable to reduce cell adhesiveness by coating the surface of the material. For example, it is possible to coat the surface of the material with the metal or metal oxide materials described above, or coat the surface of the material with a synthetic phospholipid polymer mimicking a cellular membrane (e.g., LIPIDURE available from NOF Corporation).

It is preferable that the nozzle 121 be formed as a through hole having a substantially perfect circle shape in approximately the center of the membrane 12C. In this case, the diameter of the nozzle 121 is not particularly limited but is preferably twice or more greater than the size of the fluorescent-stained cell 350 in order to prevent the nozzle 121 from being clogged with the fluorescent-stained cell 350. When the fluorescent-stained cell 350 is, for example, an animal cell, particularly, a human cell, the diameter of the nozzle 121 is preferably 10 micrometers or greater and more preferably 100 micrometers or greater in conformity with the cell used, because a human cell typically has a size of about 5 micrometers or greater but 50 micrometers or less.

On the other hand, when a liquid droplet is extremely large, it is difficult to achieve an object of forming a minute liquid droplet. Therefore, the diameter of the nozzle 121 is preferably 200 micrometers or less. That is, in the liquid droplet discharging unit 10C, the diameter of the nozzle 121 is typically in the range of 10 micrometers or greater but 200 micrometers or less.

The driving element 13C is formed on the lower surface of the membrane 12C. The shape of the driving element 13C can be designed to match the shape of the membrane 12C. For example, when the planar shape of the membrane 12C is a circular shape, it is preferable to form a driving element 13C having an annular (ring-like) planar shape around the nozzle 121. The driving method for driving the driving element 13C may be the same as the driving method for driving the driving element 13.

The driving unit 20 can selectively (for example, alternately) apply to the driving element 13C, a discharging waveform for vibrating the membrane 12C to form a liquid droplet 310 and a stirring waveform for vibrating the membrane 12C to an extent until which a liquid droplet 310 is not formed.

For example, the discharging waveform and the stirring waveform may both be rectangular waves, and the driving voltage for the stirring waveform may be set lower than the driving voltage for the discharging waveform. This makes it possible for a liquid droplet 310 not to be formed by application of the stirring waveform. That is, it is possible to control the vibration state (degree of vibration) of the membrane 12C depending on whether the driving voltage is high or low.

In the liquid droplet discharging unit 10C, the driving element 13C is formed on the lower surface of the membrane 12C. Therefore, when the membrane 12 is vibrated by means of the driving element 13C, a flow can be generated in a direction from the lower portion to the upper portion in the liquid chamber 11C.

Here, the fluorescent-stained cells 350 move upward from lower positions, to generate a convection current in the liquid chamber 11C to stir the cell suspension 300 containing the fluorescent-stained cells 350. The flow from the lower portion to the upper portion in the liquid chamber 11C disperses the settled, aggregated fluorescent-stained cells 350 uniformly in the liquid chamber 11C.

That is, by applying the discharging waveform to the driving element 13C and controlling the vibration state of the membrane 12C, the driving unit 20 can cause the cell suspension 300 retained in the liquid chamber 11C to be discharged through the nozzle 121 in the form of a liquid droplet 310. Further, by applying the stirring waveform to the driving element 13C and controlling the vibration state of the membrane 12C, the driving unit 20 can stir the cell suspension 300 retained in the liquid chamber 11C. During stirring, no liquid droplet 310 is discharged through the nozzle 121.

In this way, stirring the cell suspension 300 while no liquid droplet 310 is being formed can prevent settlement and aggregation of the fluorescent-stained cells 350 over the membrane 12C and can disperse the fluorescent-stained cells 350 in the cell suspension 300 without unevenness. This can suppress clogging of the nozzle 121 and variation in the number of fluorescent-stained cells 350 in the liquid droplets 310 to be discharged. This makes it possible to stably discharge the cell suspension 300 containing the fluorescent-stained cells 350 in the form of liquid droplets 310 continuously for a long time.

In the liquid droplet forming device 401C, bubbles may mix in the cell suspension 300 in the liquid chamber 11C. Also in this case, with the atmospherically exposed portion 115 provided at the top of the liquid chamber 11C, the liquid droplet forming device 401C can be evacuated of the bubbles mixed in the cell suspension 300 to the outside air through the atmospherically exposed portion 115. This enables continuous, stable formation of liquid droplets 310 without a need for disposing of a large amount of the liquid for bubble evacuation.

That is, the discharging state is affected when mixed bubbles are present at a position near the nozzle 121 or when many mixed bubbles are present over the membrane 12C. Therefore, in order to perform stable formation of liquid droplets for a long time, there is a need for eliminating the mixed bubbles. Typically, mixed bubbles present over the membrane 12C move upward autonomously or by vibration of the membrane 12C. Because the liquid chamber 11C is provided with the atmospherically exposed portion 115, the mixed bubbles can be evacuated through the atmospherically exposed portion 115. This makes it possible to prevent occurrence of empty discharging even when bubbles mix in the liquid chamber 11C, enabling continuous, stable formation of liquid droplets 310.

At a timing at which a liquid droplet is not being formed, the membrane 12C may be vibrated to an extent until which a liquid droplet is not formed, in order to positively move the bubbles upward in the liquid chamber 11C.

—Electric or Magnetic Detection Method—

In the case of the electric or magnetic detection method, as illustrated in FIG. 25, a coil 200 configured to count the number of cells is installed as a sensor immediately below a discharging head configured to discharge the cell suspension onto a plate 700′ from a liquid chamber 11′ in the form of a liquid droplet 310′. Cells are coated with magnetic beads that are modified with a specific protein and can adhere to the cells. Therefore, when the cells to which magnetic beads adhere pass through the coil, an induced current is generated to enable detection of presence or absence of the cells in the flying liquid droplet. Generally, cells have proteins specific to the cells on the surfaces of the cells. Modification of magnetic beads with antibodies that can adhere to the proteins enables adhesion of the magnetic beads to the cells. As such magnetic beads, a ready-made product can be used. For example, DYNABEADS (registered trademark) available from Veritas Corporation can be used.

<Operation for Observing Cells Before Discharging>

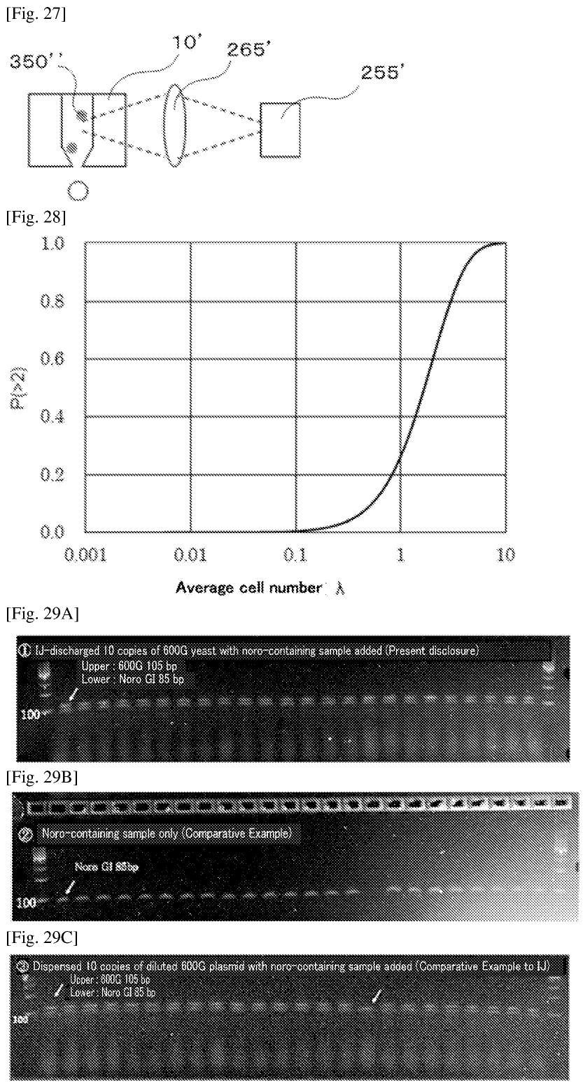

The operation for observing cells before discharging may be performed by, for example, a method for counting cells 350′ that have passed through a micro-flow path 250 illustrated in FIG. 26 or a method for capturing an image of a portion near a nozzle portion of a discharging head illustrated in FIG. 27. The method of FIG. 26 is a method used in a cell sorter device, and, for example, CELL SORTER SH800Z available from Sony Corporation can be used. In FIG. 26, a light source 260 emits laser light into the micro-flow path 250, and a detector 255 detects scattered light or fluorescence through a condenser lens 265. This enables discrimination of presence or absence of cells or the kind of the cells, while a liquid droplet is being formed. Based on the number of cells that have passed through the micro-flow path 250, this method enables estimation of the number of cells that have landed in a predetermined well.

As the discharging head 10′ illustrated in FIG. 27, a single cell printer available from Cytena GmbH can be used. In FIG. 27, it is possible to estimate the number of cells that have landed in a predetermined well, by capturing an image of the portion near the nozzle portion with an image capturing unit 255′ through a lens 265′ before discharging and estimating based on the captured image that cells 350″ present near the nozzle portion have been discharged, or by estimating the number of cells that are considered to have been discharged based on a difference between images captured before and after discharging. The method of FIG. 27 is more preferable because the method enables on-demand liquid droplet formation, whereas the method of FIG. 26 for counting cells that have passed through the micro-flow path generates liquid droplets continuously.

<Operation for Counting Cells after Landing>

The operation for counting cells after landing may be performed by a method for detecting fluorescent-stained cells by observing the wells in the plate with, for example, a fluorescence microscope. This method is described in, for example, Sangjun et al., PLoS One, Volume 6(3), e17455.

Methods for observing cells before discharging a liquid droplet or after landing have the problems described below. Depending on the kind of the plate to be produced, it is the most preferable to observe cells in a liquid droplet that is being discharged. In the method for observing cells before discharging, the number of cells that are considered to have landed is counted based on the number of cells that have passed through a flow path and image observation before discharging (and after discharging). Therefore, it is not confirmed whether the cells have actually been discharged, and an unexpected error may occur. For example, there may be a case where because the nozzle portion is stained, a liquid droplet is not discharged appropriately but adheres to the nozzle plate, thus failing to make the cells in the liquid droplet land. Moreover, there may occur a problem that the cells stay behind in a narrow region of the nozzle portion, or a discharging operation causes the cells to move beyond assumption and go outside the range of observation.

The method for detecting cells on the plate after landing also have problems. First, there is a need for preparing a plate that can be observed with a microscope. As a plate that can be observed, it is common to use a plate having a transparent, flat bottom surface, particularly a plate having a bottom surface formed of glass. However, there is a problem that such a special plate is incompatible with use of ordinary wells. Further, when the number of cells is large, such as some tens of cells, there is a problem that correct counting is impossible because the cells may overlap with each other. Accordingly, it is preferable to perform the operation for observing cells before discharging and the operation for counting cells after landing, in addition to counting the number of cells contained in a liquid droplet with a sensor and a particle number (cell number) counting unit after the liquid droplet is discharged and before the liquid droplet lands in a well.

As the light receiving element, a light receiving element including one or a small number of light receiving portion(s), such as a photodiode, an Avalanche photodiode, and a photomultiplier tube may be used. In addition, a two-dimensional sensor including light receiving elements in a two-dimensional array formation, such as a CCD (Charge Coupled Device), a CMOS (Complementary Metal Oxide Semiconductor), and a gate CCD may be used.

When using a light receiving element including one or a small number of light receiving portion(s), it is conceivable to determine the number of cells contained, based on the fluorescence intensity, using a calibration curve prepared beforehand. Here, binary detection of whether cells are present or absent in a flying liquid droplet is common. When the cell suspension is discharged in a state that the cell concentration is so sufficiently low that almost only 1 or 0 cell(s) will be contained in a liquid droplet, sufficiently accurate counting is available by the binary detection. On the premise that cells are randomly distributed in the cell suspension, the cell number in a flying liquid droplet is considered to conform to a Poisson distribution, and the probability P (>2) at which two or more cells are contained in a liquid droplet is represented by a formula (1) below. FIG. 28 is a graph plotting a relationship between the probability P (>2) and an average cell number. Here, X is a value representing an average cell number in a liquid droplet and obtained by multiplying the cell concentration in the cell suspension by the volume of a liquid droplet discharged.

P(>2)=1−(1+λ)×e^{-λ} formula (1)

When performing cell number counting by binary detection, in order to ensure accuracy, it is preferable that the probability P (>2) be a sufficiently low value, and that λ satisfy: λ<0.15, at which the probability P (>2) is 1% or lower. The light source is not particularly limited and may be appropriately selected depending on the intended purpose, so long as the light source can excite fluorescence from cells. It is possible to use, for example, an ordinary lamp such as a mercury lamp and a halogen lamp to which a filter is applied for emission of a specific wavelength, a LED (Light Emitting Diode), and a laser. However, particularly when forming a minute liquid droplet of 1 nL or less, there is a need for irradiating a small region with a high light intensity. Therefore, use of a laser is preferable. As a laser light source, various commonly known lasers such as a solid-state laser, a gas laser, and a semiconductor laser can be used. The excitation light source may be a light source that is configured to continuously irradiate a region through which a liquid droplet passes or may be a light source that is configured for pulsed irradiation in synchronization with discharging of a liquid droplet at a timing delayed by a predetermined period of time from the operation for discharging the liquid droplet.

<<Step of Calculating Degrees of Certainty of Estimated Numbers of Nucleic Acids in Cell Suspension Producing Step, Liquid Droplet Landing Step, and Cell Number Counting Step>>

The step of calculating the degrees of certainty of estimated numbers of nucleic acids in the cell suspension producing step, the liquid droplet landing step, and the cell number counting step is a step of calculating the degree of certainty in each of the cell suspension producing step, the liquid droplet landing step, and the cell number counting step.

The degree of certainty of an estimated number of nucleic acids can be calculated in the same manner as calculating the degree of certainty in the cell suspension producing step.

The timing at which the degrees of certainty are calculated may be collectively in the next step to the cell number counting step, or may be at the end of each of the cell suspension producing step, the liquid droplet landing step, and the cell number counting step in order for the degrees of certainty to be synthesized in the next step to the cell number counting step. In other words, the degrees of certainty in these steps need only to be calculated at arbitrary timings by the time when synthesis is performed.

<<Outputting Step>>

The outputting step is a step of outputting a counted value of the number of cells contained in the cell suspension that has landed in a well, counted by a cell number counting unit based on a detection result measured by a sensor.

The counted value means a number of cells contained in the well, calculated by the cell number counting unit based on the detection result measured by the sensor.

Outputting means sending a value counted by a device such as a motor, communication equipment, and a calculator upon reception of an input to an external server serving as a count result memory unit in the form of electronic information, or printing the counted value as a printed matter.

In the outputting step, an observed value or an estimated value obtained by observing or estimating the number of cells or the number of nucleic acids in each well of a plate during production of the plate is output to an external memory unit.

Outputting may be performed at the same time as the cell number counting step, or may be performed after the cell number counting step.

<<Recording Step>>

The recording step is a step of recording the observed value or the estimated value output in the outputting step.

The recording step can be suitably performed by a recording unit.

Recording may be performed at the same time as the outputting step, or may be performed after the outputting step.

Recording means not only supplying information to a recording medium but also storing information in a memory unit.

<<Nucleic Acid Extracting Step>>

The nucleic acid extracting step is a step of extracting nucleic acids from cells in the well.

Extracting means destroying, for example, cellular membranes and cell walls to pick out nucleic acids.

As the method for extracting nucleic acids from cells, there is known a method of thermally treating cells at from 90 degrees C. through 100 degrees C. By a thermal treatment at 90 degrees C. or lower, there is a possibility that DNA may not be extracted. By a thermal treatment at 100 degrees C. or higher, there is a possibility that DNA may be decomposed. Here, it is preferable to perform thermal treatment with addition of a surfactant.

The surfactant is not particularly limited and may be appropriately selected depending on the intended purpose. Examples of the surfactant include ionic surfactants and nonionic surfactants. One of these surfactants may be used alone or two or more of these surfactants may be used in combination. Among these surfactants, nonionic surfactants are preferable because proteins are neither modified nor deactivated by nonionic surfactants, although depending on the addition amount of the nonionic surfactants.

Examples of the ionic surfactants include fatty acid sodium, fatty acid potassium, alpha-sulfo fatty acid ester sodium, sodium straight-chain alkyl benzene sulfonate, alkyl sulfuric acid ester sodium, alkyl ether sulfuric acid ester sodium, and sodium alpha-olefin sulfonate. One of these ionic surfactants may be used alone or two or more of these ionic surfactants may be used in combination. Among these ionic surfactants, fatty acid sodium is preferable and sodium dodecyl sulfate (SDS) is more preferable.

Examples of the nonionic surfactants include alkyl glycoside, alkyl polyoxyethylene ether (e.g., BRIJ series), octyl phenol ethoxylate (e.g., TRITON X series, IGEPAL CA series, NONIDET P series, and NIKKOL OP series), polysorbates (e.g., TWEEN series such as TWEEN 20), sorbitan fatty acid esters, polyoxyethylene fatty acid esters, alkyl maltoside, sucrose fatty acid esters, glycoside fatty acid esters, glycerin fatty acid esters, propylene glycol fatty acid esters, and fatty acid monoglyceride. One of these nonionic surfactants may be used alone or two or more of these nonionic surfactants may be used in combination. Among these nonionic surfactants, polysorbates are preferable.

The content of the surfactant is preferably 0.01% by mass or greater but 5.00% by mass or less relative to the total amount of the cell suspension in the well. When the content of the surfactant is 0.01% by mass or greater, the surfactant can be effective for DNA extraction. When the content of the surfactant is 5.00% by mass or less, inhibition against amplification can be prevented during PCR. As a numerical range in which both of these effects can be obtained, the range of 0.01% by mass or greater but 5.00% by mass or less is preferable.

The method described above may not be able to sufficiently extract DNA from a cell that has a cell wall. Examples of methods for such a case include an osmotic shock procedure, a freeze-thaw method, an enzymic digestive method, use of a DNA extraction kit, an ultrasonic treatment method, a French press method, and a homogenizer method. Among these methods, an enzymic digestive method is preferable because the method can save loss of extracted DNA.

<<Other Steps>>

The other steps are not particularly limited and may be appropriately selected depending on the intended purpose. Examples of the other steps include an enzyme deactivating step.

—Enzyme Deactivating Step—

The enzyme deactivating step is a step of deactivating an enzyme.

Examples of the enzyme include DNase, RNase, and an enzyme used in the nucleic acid extracting step in order to extract a nucleic acid.

The method for deactivating an enzyme is not particularly limited and may be appropriately selected depending on the intended purpose. A known method can be suitably used.

The device of the present disclosure is widely used in, for example, biotechnology-related industries, life science industries, and health care industries, and can be used suitably for, for example, equipment configuration or generation of calibration curves and management of the accuracy of a testing device.

In the case of working the device for infectious diseases, the device is applicable to methods stipulated as official analytical methods or officially announced methods.

### EXAMPLES

The present disclosure will be described below by way of Examples. The present disclosure should not be construed as being limited to these Examples.

### Example 1

<Preparation of Nucleic Acid Sample>

—Production of Yeast Suspension for Series of Low-Concentration Nucleic Acid Samples—

—Gene Recombinant Yeast—

FOR producing a recombinant, a budding yeast YIL015W BY4741 (available from ATCC, ATCC4001408) was used as a carrier cell for one copy of a specific nucleic acid sequence.

The specific nucleic acid sequence was a DNA600-G sequence. In the form of a plasmid produced by arranging the specific nucleic acid sequence in tandem with URA3, which was a selectable marker, one copy of the specific nucleic acid sequence was introduced into yeast genome DNA by homologous recombination, targeting a BAR1 region of the carrier cell, to produce a gene recombinant yeast.

—Culturing and Cell-Cycle Control—

In an Erlenmeyer flask, a 90-mL fraction of the gene recombinant yeast cultured in 50 g/L of a YPD medium (available from Takara Bio Inc., CLN-630409) was mixed with 900 microliters of α1-MATING FACTOR ACETATE SALT (available from Sigma-Aldrich Co., LLC, T6901-5MG, hereinafter referred to as “α factor”) prepared to 500 micrograms/mL with a Dulbecco's phosphate buffered saline (available from Thermo Fisher Scientific Inc., 14190-144, hereinafter referred to as “DPBS”).

Next, the resultant was incubated with a bioshaker (available from Taitec Corporation, BR-23FH) at a shaking speed of 250 rpm at a temperature of 28 degrees C. for 2 hours, to synchronize the yeast at a G0/G1 phase, to obtain a yeast suspension.

—Fixing—

Forty-five milliliters of the synchronization-confirmed yeast suspension was transferred to a centrifuge tube (available from As One Corporation, VIO-50R) and centrifuged with a centrifugal separator (available from Hitachi, Ltd., F16RN,) at a rotation speed of 3,000 rpm for 5 minutes, with subsequent supernatant removal, to obtain yeast pellets. Four milliliters of formalin (available from Wako Pure Chemical Industries, Ltd., 062-01661) was added to the obtained yeast pellets, and the resultant was left to stand still for 5 minutes, then centrifuged with subsequent supernatant removal, and suspended with addition of 10 mL of ethanol, to obtain a fixed yeast suspension.

—Nuclear Staining—

Two hundred microliters of the fixed yeast suspension was fractionated, washed with DPBS once, and resuspended in 480 microliters of DPBS.

Next, to the resultant, 20 microliters of 20 mg/mL RNase A (available from Nippon Gene Co., Ltd., 318-06391) was added, followed by incubation with a bioshaker at 37 degrees C. for 2 hours.

Next, to the resultant, 25 microliters of 20 mg/mL proteinase K (available from Takara Bio Inc., TKR-9034) was added, followed by incubation with PETIT COOL (available from Waken B Tech Co., Ltd., PETIT COOL MINI T-C) at 50 degrees C. for 2 hours.

Finally, to the resultant, 6 microliters of 5 mM SYTOX GREEN NUCLEIC ACID STAIN (available from Thermo Fisher Scientific Inc., 57020) was added, followed by staining in a light-shielded environment for 30 minutes.

—Dispersing—

The stained yeast suspension was subjected to dispersion treatment using an ultrasonic homogenizer (available from Yamato Scientific Co., Ltd., LUH150,) at a power output of 30% for 10 seconds, to obtain a yeast suspension ink.

<Filling of Nucleic Acid Samples>

—Filling of Series of Low-Concentration Nucleic Acid Samples—

—Dispensing of Yeast Suspension with Number Counting—

—Dispensing and Cell Counting—

A plate with a known cell number was produced by counting the number of yeast cells in liquid droplets in the manner described below to discharge one cell per well. Specifically, with the use of the liquid droplet forming device illustrated in FIG. 21, the yeast suspension ink was sequentially discharged into each well of a 96 plate (product name: MICROAMP 96-WELL REACTION PLATE, available from Thermo Fisher Scientific Inc.), using a piezoelectricity applying-type discharging head (available in-house) as a liquid droplet discharging unit at 10 Hz.

An image of yeast cells in a liquid droplet discharged was captured using a high-sensitivity camera (available from Tokyo Instruments Inc., SCMOS PCO.EDGE) as a light receiving unit and using a YAG laser (available from Spectra-Physics, Inc., EXPLORER ONE-532-200-KE) as a light source, and the cell number was counted by image processing with image processing software IMAGE J serving as a particle number counting unit for the captured image. In this way, a plate with a known cell number of 1 was produced.

—Extraction of Nucleic Acids—

With a Tris-EDTA (TE) buffer and ColE1 DNA (available from Wako Pure Chemical Industries, Ltd., 312-00434), ColE1/TE was prepared at 5 ng/microliter. With ColE1/TE, a Zymolyase solution of Zymolyase(R) 100T (available from Nacalai Tesque Inc., 07665-55) was prepared at 1 mg/mL.

Four microliters of the Zymolyase solution was added into each well of the produced plate with a known cell number, incubated at 37.2 degrees C. for 30 minutes, to dissolve cell walls (extraction of nucleic acids), and then thermally treated at 95 degrees C. for 2 minutes, to produce a reference device.

Next, in order to consider the reliability of a result obtained from a plate with a known cell number, a plate with a known cell number of 1 was produced and the uncertainty for the cell number of 1 was calculated. Note that it is possible to calculate uncertainties for various copy numbers, by using the method described below for each specific copy number.

—Calculation of Uncertainty—

In the present Example, the number of cells in a liquid droplet, the copy number of amplifiable reagents in a cell, the number of cells in a well, and contamination were used as the factors for uncertainty.

As the number of cells in a liquid droplet, the number of cells in a liquid droplet, counted based on an analysis of an image of the liquid droplet discharged by a discharging unit, and the number of cells obtained based on microscopic observation of each liquid droplet landed on a glass slide among liquid droplets discharged by a discharging unit so as to be landed on the glass slide were used.

The copy number of nucleic acids in a cell (cell cycle) was calculated using the ratio of cells that were at a G1 phase of the cell cycle (99.5%) and the ratio of cells that were at a G2 phase (0.5%).

As the number of cells in a well, the number of discharged liquid droplets landed in a well was counted. However, in counting 96 samples in total, all of the samples were landed in the wells as liquid droplets. Therefore, as a factor, the number of cells in a well was excluded from calculation of the uncertainty.

To confirm contamination, a filtrate (4 microliters) of the ink was subjected to real-time PCR to see whether any other nucleic acid than the amplifiable reagents in the cell was mixed in the ink liquid. This was tried three times. The result was the limit of detection in all of the three tries. Therefore, as a factor, contamination was also excluded from calculation of the uncertainty.

For the uncertainty, standard deviation was calculated from the measured values of each factor and multiplied by a sensitivity coefficient, to obtain a standard uncertainty unified in the unit of the measured quantity. Based on such standard uncertainties, a synthesized standard uncertainty was calculated according to the sum-of-squares method. The synthesized standard uncertainty covered only the values in a range of about 68% of a normal distribution. Therefore, by doubling the synthesized standard uncertainty, it was possible to obtain an expanded uncertainty, which was an uncertainty that took into account a range of about 95% of the normal distribution. The results are presented in the budget sheet of Table 4 below.

TABLE 4

| | | | | | | | | | Standard |
|---|---|---|---|---|---|---|---|---|---|
| | | | | | | | | | uncertainty |
| | | | | | | | | | (in unit of |
| | Factors of | Value | | Probability | | Standard | | Sensitivity | measured |
| Symbol | uncertainty | (±) | | distribution | Divisor | uncertainty | | coefficient | quantity) |
| u1 | Number of cells | 0.1037 | cells | — | 1 | 0.1037 | cells | 1.0290 | 0.1067 copies |
| | in liquid droplet | | | | | | | copies/cell | |
| u2 | Number of | 0.0709 | copies | — | 1 | 0.0709 | copies | — | 0.0709 copies |
| | nucleic acid | | | | | | | | |
| | molecules in | | | | | | | | |
| | cell (cell cycle) | | | | | | | | |
| u3 | Number of cells | — | | — | — | — | | — | — |
| | in well | | | | | | | | |
| u4 | Contamination | — | | — | — | — | | — | — |
| uc | Synthesized | | | Normal | | | | | 0.1281 copies |
| | standard | | | distribution | | | | | |
| | uncertainty | | | | | | | | |
| U | Expanded | | | Normal | | | | | 0.2562 copies |
| | uncertainty | | | distribution | | | | | |
| | | | | (k = 2) | | | | | |

In Table 4, “Symbol” means an arbitrary symbol associated with a factor of the uncertainty.

In Table 4, “Value (±)” indicates an experimental standard deviation in average value, obtained by dividing a calculated experimental standard deviation by the square root of the number of data.

In Table 4, “Probability distribution” is a probability distribution of a factor of the uncertainty. The field was left blank for type-A uncertainty evaluation, whereas either normal distribution or rectangular distribution was filled in the field for type-B uncertainty evaluation. In the present Example, only type-A uncertainty evaluation was performed. Therefore, the probability distribution field was left blank.

In Table 4, “Divisor” means a number that normalizes the uncertainty of each factor.

In Table 4, “Standard uncertainty” is a value obtained by dividing “Value (±)” by “Divisor”.

In Table 4, “Sensitivity coefficient” means a value used for unification to the unit of the measured quantity.

Next, average specific copy numbers of nucleic acid samples filled in wells and uncertainties were calculated. The results are presented in Table 5. The coefficients of variation CV were calculated by dividing the uncertainty values by the average specific copy numbers.

TABLE 5

Average specific copy number

| Average | Uncertainty | Coefficient of variation CV |
|---|---|---|
| Copy | Copy | % |
| 1.02E+00 | 1.28E−01 | 12.60 |
| 2.03E+00 | 1.81E−01 | 8.91 |
| 4.07E+00 | 2.56E−01 | 6.30 |
| 8.13E+00 | 3.62E−01 | 4.46 |
| 1.63E+01 | 5.12E−01 | 3.15 |
| 2.13E+01 | 5.87E−01 | 2.75 |
| 6.50E+01 | 1.02E+00 | 1.58 |
| 1.30E+02 | 1.45E+00 | 1.11 |

According to the inkjet method, the accuracy for dispensing a specific copy number of 1 of a nucleic acid sample, i.e., one copy of a nucleic acid sample (one yeast cell) per well was found to be ±0.1281 copies. In the case of filling one or more copies per well, the accuracy at which a specific copy number of nucleic acid samples would be filled would be determined by accumulation of this accuracy.

From the results described above, the obtained expanded uncertainty was stored as data for each device as the indicator of the variation in measurement. This would enable a user to use the indicator of the uncertainty as the reference for judging the reliability of a result of measurement in each well in an experiment. Use of the reference for judging the reliability would enable highly accurate evaluation of the performance of an analytical test.

—Association of Uncertainty with Each Filled Portion—

The calculated uncertainty (or coefficient of variation) described above was associated with each well.

In this way, it was possible to calculate the average copy number of nucleic acids of the series of low-concentration nucleic acid samples and the uncertainty and the coefficient of variation of the average copy number, and associate the average copy number, the uncertainty, and the coefficient of variation with each well.

—Amplification Reaction—

A sample was filled in a sample filling well filled with the above-produced nucleic acid serving as the amplifiable reagent. Subsequently, the testing target nucleic acid and the nucleic acid serving as the amplifiable reagent were allowed to undergo amplification reactions in the same well according to a PCR method.

Through the detection result obtaining unit and the detection result analyzing unit described above, presence or absence of the testing target was determined by the determining unit based on the result of amplification of the amplifiable reagent and the result of amplification of the testing target. When the case of (1) below was applicable, a “positive” determination was made. When the case of (2) below was applicable, a “negative” determination was made.

(1) When the amplifiable reagent was amplified and the testing target was amplified, the testing target was present and the detection result was positive.

(2) When the amplifiable reagent was amplified and the testing target was not amplified, the testing target was absent or at least less than the specific copy number of the amplifiable reagent, and the detection result was negative.

### Example 2

<Negative Test for Norovirus in Shellfish>

A method for testing norovirus in a shellfish will be described below as Example.

First, the mantle of the shellfish was cut away, and fatty parts attached to the mid-intestinal gland were carefully removed. The mid-intestinal gland was put in a sampling bag for a homogenizer, and crushed with addition of from 7-fold through 10-fold PBS (−). The crushed sample was cool-centrifuged at 10,000 rpm for 20 minutes. A 30% by mass sucrose solution was poured into a centrifuge tube for ultracentrifugation in an amount of about 10% by mass of the amount of the centrifuge tube. Onto the resultant, the supernatant of the cool-centrifuged sample was calmly stratified, followed by cool centrifugation at 35,000 rpm for 180 minutes. After the liquid phase was aspirated with an aspirator, the wall of the tube was quickly washed with PBS (−). Two hundred microliters of DDW was added to the sediment to float the sediment. The resultant was used as a sample to be used for extracting virus RNA.

Next, a reverse transcription reaction was performed using SUPER SCRIPT II (available from Invitrogen). A total of 15 microliters of a reaction liquid was prepared, using the sample (7.5 microliters), a 5×SSII buffer (2.25 microliters), 10 mM dNTPs (0.75 microliters), a random primer (1.0 microgram/microliter) (0.375 microliters), a ribonuclease inhibitor (33 units/microliter) (0.5 microliters), 100 mM DTT (0.75 microliters), SUPER SCRIPT II RT (200 units/microliter) (0.75 microliters), and distilled water (2.125 microliters). The reaction liquid was incubated at 42 degrees C. for 1 hour. Next, the resultant was heated at 99 degrees C. for 5 minutes, to deactivate the enzyme. Subsequently, the resultant was left to stand still on ice.

The sample that had undergone the reverse transcription reaction was filled in an amount of 5.0 microliters in a sample filling well filled with a nucleic acid produced in the same manner as in Example 1 to serve as the amplifiable reagent. Subsequently, the testing target nucleic acid and the nucleic acid serving as the amplifiable reagent were allowed to undergo amplification reactions in the same well according to a PCR method. The composition of the reaction liquid (total of 50 microliters) contained distilled water (33.75 microliters), a 10×EX TAQ™ buffer (5.0 microliters), dNTP (2.5 mM) (4.0 microliters), a NV primer F (50 micromoles) (0.5 microliters), a NV primer R (50 micromoles) (0.5 microliters), a primer F (50 micromoles) (0.5 microliters) for amplifying the nucleic acid serving as the amplifiable reagent, a primer R (50 micromoles) (0.5 microliters) for amplifying the nucleic acid serving as the amplifiable reagent, cDNA (sample) (5.0 microliters), and EX TAQ (5 units/microliter) (0.25 microliters).

Amplification of the amplifiable reagent was performed by PCR using T100™ THERMAL CYCLER (available from Bio-Rad Laboratories, Inc.). First, the amplifiable reagent was incubated at 50 degrees C. for 2 minutes, and then 95 degrees C. for 10 minutes. Subsequently, the resultant was subjected 35 times to a temperature cycle including 2 steps, namely 95 degrees C. for 30 seconds and 61 degrees C. for 2 minutes. Finally, the resultant was incubated at 61 degrees C. for 2 minutes, and subsequently cooled to 4 degrees C., to terminate the reaction.

For the confirmation of the results, agarose electrophoresis was performed using MOTHER E-BASE™ DEVICE (available from Invitrogen™) and E-GEL™ 48 AGAROSE GELS, 4% (available from Invitrogen™). Electrophoresis was performed at 100 V for 20 minutes.

Based on the result of amplification of the testing target obtained as above, presence or absence of the testing target was determined. When the case of (1) below was applicable, a “positive” determination was made. When the case of (2) below was applicable, a “negative” determination was made.

(1) When the amplifiable reagent was amplified and the testing target was amplified, the testing target was present and the detection result was positive.

(2) When the amplifiable reagent was amplified and the testing target was not amplified, the testing target was absent or at least less than the specific copy number of the amplifiable reagent, and the detection result was negative.

In the present Example, (1) 10 cells (copies) of 600G yeast filled per well by an inkjet method (IJ) with a norovirus-containing sample added (Example of the present disclosure), (2) a norovirus-containing sample only, and (3) diluted 600G plasmid dispensed by a manual operation by an amount corresponding to 10 copies per well of a 96-well plate with a norovirus-containing sample added (Comparative Example to IJ) were prepared using 96-well plates.

FIG. 29A is a diagram illustrating a result of agarose electrophoresis of a sample (1) performed after PCR amplification of the sample in a negative test for norovirus in a shellfish in Example 2, where the sample (1) was prepared by discharging 10 cells (copies) of 600G yeast by IJ and adding a norovirus-containing sample to the resultant (Example of the present disclosure).

FIG. 29B is a diagram illustrating a result of agarose electrophoresis of a sample (2) performed after PCR amplification of the sample, where the sample (2) was prepared to contain norovirus only.

FIG. 29C is a diagram illustrating a result of agarose electrophoresis of a sample (3) performed after PCR amplification of the sample in a negative test for norovirus in a shellfish in Example 2, where the sample (3) was prepared by diluting 600G plasmid, dispensing the resultant by an amount corresponding to 10 copies per well by a manual operation, and adding a norovirus-containing sample to the resultant (Comparative Example to IJ).

In (1), both of an amplification product (105 bp, upper) of 600G and an amplification product (85 bp, lower) of Noro GI were observed as two bands as illustrated in FIG. 29A to FIG. 29C.

In (2), only an amplification product (85 bp) of Noro GI was observed as a band as illustrated in FIG. 29A to FIG. 29C. The 16th sample was not observed in the band and was negative. However, it was impossible to determine definitely whether the detection target Noro GI was actually absent in the analyte sample, i.e., whether the negative determination was correct, or whether Noro GI was actually present but erroneously determined as absent (negative) due to failure of identifying, i.e., whether the determination was false-negative.

In (3), the determining method of the present disclosure was tried using a positive control sample produced by dilution. As illustrated in FIG. 29A to FIG. 29C, the 16th sample of the positive control sample was not observed in the band of a 600G amplification product (105 bp). This was an Example in which the positive control sample became false-negative due to variation caused by dilution. This means that in order to carry out the determining method of the present disclosure, it is preferable to have a high filling accuracy presented in the present disclosure.

Aspects of the present disclosure are as follows, for example.

<1> A detection determining method used in detection of a testing target in a sample by amplification of the testing target and an amplifiable reagent, where the amplifiable reagent is provided in a specific copy number of 200 or less, the detection determining method including

determining that the testing target is present and a detection result is positive when the amplifiable reagent is amplified and the testing target is amplified, and determining that the testing target is absent or at least less than the specific copy number of the amplifiable reagent and a detection result is negative when the amplifiable reagent is amplified and the testing target is not amplified.

<2> The detection determining method according to <1>,

wherein the amplifiable reagent is a nucleic acid.

<3> The detection determining method according to <2>,

wherein the nucleic acid that is the amplifiable reagent is incorporated in a nucleic acid in a nucleus of a cell.

<4> The detection determining method according to <3>,

wherein the cell is a yeast cell.

<5> The detection determining method according to any one of <1> to <4>,

wherein a limit of detection of the testing target and a limit of detection of the amplifiable reagent are comparable to each other.

<6> The detection determining method according to any one of <1> to <5>,

wherein the amplifiable reagent is filled in a sample filling well to be filled with a sample, and the testing target and the amplifiable reagent are amplified in a same sample filling well.

<7> The detection determining method according to any one of <2> to <6>,

wherein a base sequence of the testing target and a base sequence of the amplifiable reagent are different from each other.

<8> The detection determining method according to <7>,

wherein a positive control having a base sequence same as the base sequence of the testing target is filled in a certain amount in a different well from the sample filling well and allowed to undergo an amplification treatment.

<9> The detection determining method according to <8>,

wherein the detection determining method is used for genetic testing in which the testing target is a virus, a bacterium, or animal species determination of edible meat.

<10> The detection determining method according to any one of <1> to <9>,

wherein the specific copy number of the amplifiable reagent is a known number.

<11> A detection determining device used in detection of a testing target in a sample by amplification of the testing target and an amplifiable reagent, wherein the amplifiable reagent is provided in a number of 200 or less, the detection determining device including

a determining unit configured to determine that the testing target is present and a detection result is positive when the amplifiable reagent is amplified and the testing target is amplified, and determine that the testing target is absent or at least less than the specific copy number of the amplifiable reagent and a detection result is negative when the amplifiable reagent is amplified and the testing target is not amplified.

<12> A detection determining program used in detection of a testing target in a sample by amplification of the testing target and an amplifiable reagent, wherein the amplifiable reagent is provided in a number of 200 or less, the detection determining program causing a computer to execute a process including

determining that the testing target is present and a detection result is positive when the amplifiable reagent is amplified and the testing target is amplified, and determining that the testing target is absent or at least less than the specific copy number of the amplifiable reagent and a detection result is negative when the amplifiable reagent is amplified and the testing target is not amplified.

<13> A device used for the detection determining method according to any one of <1> to <10>, the device including

at least one sample filling well to be filled with a sample,

wherein the at least one sample filling well further includes an amplifiable reagent in a specific copy number, and

wherein the specific copy number of the amplifiable reagent is 200 or less.

<14> The device according to <13>,

wherein a coefficient of variation CV for the specific copy number and an average specific copy number x of the amplifiable reagent satisfy a relationship: CV</√x.

<15> The device according to <13> or <14>,

wherein the specific copy number of the amplifiable reagent is 100 or less.

<16> The device according to any one of <13> to <15>,

wherein the sample filling well includes information on the specific copy number of the amplifiable reagent and uncertainty of the specific copy number of the amplifiable reagent.

<17> The device according to any one of <13> to <16>, further including

a sealing member configured to seal an opening of the at least one sample filling well.

<18> The device according to any one of <13> to <17>,

wherein the amplifiable reagent is a nucleic acid.

<19> The device according to <18>,

wherein the nucleic acid is incorporated in a nucleic acid in a nucleus of a cell.

<20> The device according to <19>,

wherein the cell is a yeast cell.

<21> The device according to any one of <18> to <20>,

wherein a base sequence of a testing target and a base sequence of the amplifiable reagent are different from each other.

<22> The device according to any one of <13> to <21>,

wherein the at least one sample filling well includes a pair of primers for amplifying a testing target and a pair of primers for amplifying the amplifiable reagent.

<23> A device used for the detection determining method according to any one of <1> to <10>.

The detection determining method according to any one of <1> to <10>, the detection determining device according to <11>, the detection determining program according to <12>, and the device according to any one of <13> to <23> can solve the various problems in the related art and achieve the object of the present disclosure.

### REFERENCE SIGNS LIST

1: device
2: base material
3: well
4: amplifiable reagent
5: sealing member

1. A detection determining method used in detection of a testing target in a sample by amplification of the testing target and an amplifiable reagent, wherein the amplifiable reagent is provided in a specific copy number of 200 or less, the detection determining method comprising
determining that the testing target is present and a detection result is positive when the amplifiable reagent is amplified and the testing target is amplified, and
determining that the testing target is absent or at least less than the specific copy number of the amplifiable reagent and a detection result is negative when the amplifiable reagent is amplified and the testing target is not amplified.

2. The detection determining method according to claim 1, wherein
the amplifiable reagent is a nucleic acid.

3. The detection determining method according to claim 1, wherein the amplifiable reagent is filled in a sample filling well to be filled with a sample, and the testing target and the amplifiable reagent are amplified in a same sample filling well.

4. The detection determining method according to claim 2, wherein a base sequence of the testing target and a base sequence of the amplifiable reagent are different from each other.

5. The detection determining method according to claim 4, wherein a positive control having a base sequence same as the base sequence of the testing target is filled in a certain amount in a different well from the sample filling well and allowed to undergo an amplification treatment.

6. The detection determining method according to claim 5, wherein the detection determining method is used for genetic testing in which the testing target is a virus, a bacterium, or animal species determination of edible meat.

7. The detection determining method according to claim 1, wherein the specific copy number of the amplifiable reagent is a known number.

8. A detection determining device used in detection of a testing target in a sample by amplification of the testing target and an amplifiable reagent, wherein the amplifiable reagent is provided in a number of 200 or less, the detection determining device comprising
a determining unit configured to determine that the testing target is present and a detection result is positive when the amplifiable reagent is amplified and the testing target is amplified, and determine that the testing target is absent or at least less than the specific copy number of the amplifiable reagent and a detection result is negative when the amplifiable reagent is amplified and the testing target is not amplified.

9. A non-transitory recording medium storing a detection determining program used in detection of a testing target in a sample by amplification of the testing target and an amplifiable reagent, wherein the amplifiable reagent is provided in a number of 200 or less, the detection determining program causing a computer to execute a process comprising
determining that the testing target is present and a detection result is positive when the amplifiable reagent is amplified and the testing target is amplified, and
determining that the testing target is absent or at least less than the specific copy number of the amplifiable reagent and a detection result is negative when the amplifiable reagent is amplified and the testing target is not amplified.

10. A device used for the detection determining method according to claim 1, the device comprising at least one sample filling well to be filled with a sample,
wherein the at least one sample filling well comprises an amplifiable reagent in a specific copy number, and
wherein the specific copy number of the amplifiable reagent is 200 or less.

11. The device according to claim 10, wherein a base sequence of a testing target and a base sequence of the amplifiable reagent are different from each other.

12. The device according to claim 11, wherein the at least one sample filling well comprises a pair of primers for amplifying the testing target and a pair of primers for amplifying the amplifiable reagent.

13. A device used for the detection determining method according to claim 1.

14. The detection determining method according to claim 2, wherein the nucleic acid is incorporated in a nucleic acid in a nucleus of a cell.

15. The device according to claim 10, wherein a coefficient of variation CV for the specific copy number and an average specific copy number x of the amplifiable reagent satisfy a relationship: CV</√x.

16. The device according to claim 10, wherein the specific copy number of the amplifiable reagent is 100 or less.

17. The device according to claim 10, wherein the sample filling well comprises information on the specific copy number of the amplifiable reagent and uncertainty of the specific copy number of the amplifiable reagent.

18. The device according to claim 10, further comprising a sealing member configured to seal an opening of the at least one sample filling well.

19. The device according to claim 10, wherein the amplifiable reagent is a nucleic acid.

20. The device according to claim 19, wherein the nucleic acid is incorporated in a nucleic acid in a nucleus of a cell.


SEQUENCE LISTING (3 sequences)

SEQ ID NO: 1 - DNA, 600 bp, Artificial Sequence
  Synthetic nucleotides
attcgaagggtgattggatcggagataggatgggtcaatcgtagggacaatcgaagccagaatgcaagggtcaatggtacgcagaatggatggcacttagctagccagttaggatccgactatccaagcgtgtatcgtacggtgtatgcttcggagtaacgatcgcactaagcatggctcaatcctaggctgataggttcgcacatagcatgccacatacgatccgtgattgctagcgtgattcgtaccgagaactcacgccttatgactgcccttatgtcaccgcttatgtctcccgagatcacacccgttatctcagccctaatctctgcggtttagtctggccttaatccatgcctcatagctaccctcataccatcgctcataccttccgacattgcatccgtcattccaaccctgattcctacggtctaacctagcctctatcctacccagttaggttgcctcttagcatccctgttacgtacgctcttaccatgcgtcttaccttggcactatcgatgggagtatggtagcgagtatggaacggactaacgtaggcagtaagctagggtgtaaggttgggactaaggatgccag

SEQ ID NO: 2 - DNA, 85 bp, Norwalk virus(G1)
cgctggatgcgcttccatgacctcggattgtggacaggagatcgcgatcttctgcccgaattcgtaaatgatgatggcgtctaag

SEQ ID NO: 3 - DNA, 98 bp, Norwalk virus(G2)
caagagccaatgttcagatggatgagattctcagatctgagcacgtgggagggcgatcgcaatctggctcccagctttgtgaatgaagatggcgtcga